SEQUENCE LISTING (28 sequences)

SEQ ID NO: 1 - protein, 118 aa, Artificial Sequence
  Humanized D5 antibody heavy chain variable domain (HA)
QVQLVQSGAEVKKPGASVKVSCKASGYTFTDTYIHWVRQAPGQRLEWIGKIDPANGNTKYDPKFQDRVTITRDTSASTAYMELSSLRSEDTAVYYCASSNYWFDFDYWGQGTLVTVSS

SEQ ID NO: 2 - protein, 112 aa, Artificial Sequence
  Humanized D5 antibody light chain variable domain (KA)
DIVMTQSPLSLPVTPGEPASISCRSSQSIVHSNGNTYLEWYLQKPGQSPQLLIYKVSNRFSGVPDRFSGSGSGTDFTLKISRVEAEDVGVYYCYQGSHVPYTFGGGTKVEIK

SEQ ID NO: 3 - protein, 118 aa, Artificial Sequence
  Humanized D5 antibody VH domain (HM)
QVQLVQSGAEVKKPGASVKVSCTASGFNIKDTYIHWVRQAPGQRLEWIGKIDPANGNTKYDPKFQDKATITADTSANTAYMELSSLRSEDTAVYYCANSNYWFDFDYWGQGTLVTVSS

SEQ ID NO: 4 - protein, 118 aa, Artificial Sequence
  Humanized D5 antibody VH domain (HN)
QVQLVQSGAEVKKPGASVKVSCKASGFNIKDTYIHWVKQAPGQRLEWIGKIDPANGNTKYDPKFQDKATITADTSASTAYMELSSLRSEDTAVYYCANSNYWFDFDYWGQGTLVTVSS

SEQ ID NO: 5 - protein, 118 aa, Artificial Sequence
  Humanized D5 antibody heavy chain (HP)
QVQLVQSGAEVKKPGASVKVSCKASGYTFTDTYIHWVRQAPGQRLEWIGKIDPANGNTKYDPKFQDKVTITRDTSASTAYMELSSLRSEDTAVYYCANSNYWFDFDYWGQGTLVTVSS

SEQ ID NO: 6 - protein, 118 aa, Artificial Sequence
  Humanized D5 antibody VH domain (HQ)
QVQLVQSGAEVKKPGASVKVSCTASGFNIKDTYIHWVKQAPGQRLEWIGKIDPANGNTKYDPKFQDKATITADTSANTAYMELSSLRSEDTAVYYCANSNYWFDFDYWGQGTLVTVSS

SEQ ID NO: 7 - protein, 112 aa, Artificial Sequence
  Humanized D5 antibody variable domain (KB)
DVLMTQSPLSLPVTPGEPASISCRSSQSIVHSNGNTYLEWYLQKPGQSPQLLIYKVSNRFSGVPDRFSGSGSGTDFTLKISRVEAEDVGVYYCYQGSHVPYTFGGGTKVEIK

SEQ ID NO: 8 - protein, 112 aa, Artificial Sequence
  Humanized D5 antibody variable domain (KC)
DVVMTQSPLSLPVTPGEPASISCRSSQSIVHSNGNTYLEWYLQKPGQSPQLLIYKVSNRFSGVPDRFSGSGSGTDFTLKISRVEAEDVGVYYCYQGSHVPYTFGGGTKVEIK

SEQ ID NO: 9 - protein, 112 aa, Artificial Sequence
  Humanized D5 antibody variable domain (KD)
DILMTQSPLSLPVTPGEPASISCRSSQSIVHSNGNTYLEWYLQKPGQSPQLLIYKVSNRFSGVPDRFSGSGSGTDFTLKISRVEAEDVGVYYCYQGSHVPYTFGGGTKVEIK

SEQ ID NO: 10 - protein, 112 aa, Artificial Sequence
  Humanized D5 antibody variable domain (KE)
DVLMTQSPLSLPVTPGEPASISCRSSQSIVHSNGNTYLEWYLQKPGQSPKLLIYKVSNRFSGVPDRFSGSGSGTDFTLKISRVEAEDVGVYYCYQGSHVPYTFGGGTKVEIK

SEQ ID NO: 11 - protein, 118 aa, Artificial Sequence
  Humanized D5 antibody variant (HB)
QVQLVQSGAEVKKPGASVKVSCKASGFNIKDTYIHWVRQAPGQRLEWIGKIDPANGNTKYDPKFQDKATITADTSASTAYMELSSLRSEDTAVYYCANSNYWFDFDYWGQGTLVTVSS

SEQ ID NO: 12 - protein, 15 aa, Artificial Sequence
  SP70 binding epitope
YPTFGEHKQEKDLEY

SEQ ID NO: 13 - protein, 118 aa, Artificial Sequence
  Humanized D5 antibody heavy chain variant (HC)
QVQLVQSGAEVKKPGASVKVSCKASGFTFTDTYIHWVRQAPGQRLEWIGKIDPANGNTKYDPKFQDRVTITRDTSASTAYMELSSLRSEDTAVYYCASSNYWFDFDYWGQGTLVTVSS

SEQ ID NO: 14 - protein, 118 aa, Artificial Sequence
  Humanized D5 antibody heavy chain variant (HD)
QVQLVQSGAEVKKPGASVKVSCKASGYNFTDTYIHWVRQAPGQRLEWIGKIDPANGNTKYDPKFQDRVTITRDTSASTAYMELSSLRSEDTAVYYCASSNYWFDFDYWGQGTLVTVSS

SEQ ID NO: 15 - protein, 118 aa, Artificial Sequence
  Humanized D5 antibody heavy chain variant (HE)
QVQLVQSGAEVKKPGASVKVSCKASGYTITDTYIHWVRQAPGQRLEWIGKIDPANGNTKYDPKFQDRVTITRDTSASTAYMELSSLRSEDTAVYYCASSNYWFDFDYWGQGTLVTVSS

SEQ ID NO: 16 - protein, 118 aa, Artificial Sequence
  Humanized D5 antibody heavy chain variant (HF)
QVQLVQSGAEVKKPGASVKVSCKASGYTFKDTYIHWVRQAPGQRLEWIGKIDPANGNTKYDPKFQDRVTITRDTSASTAYMELSSLRSEDTAVYYCASSNYWFDFDYWGQGTLVTVSS

SEQ ID NO: 17 - protein, 118 aa, Artificial Sequence
  Humanized D5 antibody heavy chain variant (HG)
QVQLVQSGAEVKKPGASVKVSCKASGYTFTDTYIHWVRQAPGQRLEWIGKIDPANGNTKYDPKFQDKVTITRDTSASTAYMELSSLRSEDTAVYYCASSNYWFDFDYWGQGTLVTVSS

SEQ ID NO: 18 - protein, 118 aa, Artificial Sequence
  Humanized D5 antibody heavy chain variant (HH)
QVQLVQSGAEVKKPGASVKVSCKASGYTFTDTYIHWVRQAPGQRLEWIGKIDPANGNTKYDPKFQDRATITRDTSASTAYMELSSLRSEDTAVYYCASSNYWFDFDYWGQGTLVTVSS

SEQ ID NO: 19 - protein, 118 aa, Artificial Sequence
  Humanized D5 antibody heavy chain variant (HI)
QVQLVQSGAEVKKPGASVKVSCKASGYTFTDTYIHWVRQAPGQRLEWIGKIDPANGNTKYDPKFQDRVTITADTSASTAYMELSSLRSEDTAVYYCASSNYWFDFDYWGQGTLVTVSS

SEQ ID NO: 20 - protein, 118 aa, Artificial Sequence
  Humanized D5 antibody heavy chain variant (HJ)
QVQLVQSGAEVKKPGASVKVSCKASGYTFTDTYIHWVRQAPGQRLEWIGKIDPANGNTKYDPKFQDRVTITRDTSASTAYMELSSLRSEDTAVYYCANSNYWFDFDYWGQGTLVTVSS

SEQ ID NO: 21 - DNA, 354 bp, Artificial Sequence
  Nucleic acid encoding antibody polypeptide chain
caggtgcagctggtccagtcaggagcagaagtcaaaaagcccggagcatcagtcaaagtctcttgtaaagcctcaggatacacattcaccgacacatacatccactgggtgaggcaggctccaggacagcgactggagtggatcggcaagattgatcccgccaacgggaatactaagtacgaccctaaattccaggatcgggtgactattaccagagacacctccgcctctacagcttatatggagctgagctccctgcgaagcgaagatacagcagtctactattgcgcctctagtaactactggttcgactttgattattggggccaggggacactggtgactgtctcaagc

SEQ ID NO: 22 - DNA, 354 bp, Artificial Sequence
  Nucleic acid encoding antibody polypeptide chain
caggtccagctggtgcagagcggagccgaagtcaaaaagcccggagcctcagtcaaagtgtcttgtaccgcatcaggattcaacatcaaagacacctacatccactgggtgcggcaggcaccaggacagagactggagtggatcggcaagattgatcccgctaacgggaatacaaagtacgaccctaaattccaggataaggcaaccattacagccgacacatcagccaacactgcttatatggagctgagctccctgaggtccgaagataccgctgtctactattgcgcaaacagtaattactggttcgactttgattattggggccaggggactctggtgaccgtctctagt

SEQ ID NO: 23 - DNA, 336 bp, Artificial Sequence
  Nucleic acid encoding antibody polypeptide chain
gacattgtgatgacccagtcccctctgagcctgcccgtgacccccggcgaacctgcctccatttcctgccgatccagccagtccattgtgcacagcaacggaaatacatacctggagtggtatctgcagaagcccggccagtcccctcagctgctgatctacaaagtgagtaaccggttctcaggagtcccagaccggttcagcggctccgggtctggaaccgatttcacactgaagatttctagggtggaggccgaagacgtgggcgtctactattgctaccaggggagccatgtgccctatacttttggcgggggaaccaaggtcgaaatcaaa

SEQ ID NO: 24 - protein, 118 aa, Artificial Sequence
  Humanized D5 heavy chain variable domain (mD5 VH)
EVQLQQSGAELVKPGASVKLSCTASGFNIKDTYIHWVKQRPEQGLEWIGKIDPANGNTKYDPKFQDKATITADTSSNTAYLQLSSLTSEDTAVYYCANSNYWFDFDYWGQGTTLTVSS

SEQ ID NO: 25 - protein, 118 aa, Artificial Sequence
  Humanized D5 heavy chain variable domain (EF178053)
QVQLVQSGAEVKKPGASVKVSCKASGYTFTSYAMHWVRQAPGQRLEWIGWINAGNGNTKYSQKFQGRVTITRDTSASTAYMELSSLRSEDTAVYYCASGQWLAPFDYWGQGTLVTVSS

SEQ ID NO: 26 - protein, 118 aa, Artificial Sequence
  Humanized D5 heavy chain variable domain (D5_HO)
QVQLVQSGAEVKKPGASVKVSCKASGYTFTDTYIHWVKQAPGQRLEWIGKIDPANGNTKYDPKFQDRVTITRDTSASTAYMELSSLRSEDTAVYYCASSNYWFDFDYWGQGTLVTVSS

SEQ ID NO: 27 - protein, 112 aa, Artificial Sequence
  Humanized D5 anitbody light chain variable domain (D5 VK)
DVLMTQTPLSLPVSLGDQASISCRSSQSIVHSNGNTYLEWYLQKPGQSPKLLIYKVSNRFSGVPDRFSGSGSGTDFTLKISRVEADDVGVYYCYQGSHVPYTFGGGTKLEIK

SEQ ID NO: 28 - protein, 112 aa, Artificial Sequence
  Humanized D5 antibody light chain variable domain (AJ388646)
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

(19) United States
(12) Patent Application Publication
(10) Pub. No.: US 2020/0317753 A1
(43) Pub. Date: Oct. 8, 2020

(54) Humanized Antibodies Against Enterovirus 71

(71) Applicant: LIFEARC, London, Greater London (GB)

(72) Inventors: Tilbe CREIGH-PULATMEN, Stevenage (GB); Seema PATEL, Stevenage (GB); David John MATTHEWS, Stevenage (GB); Zhong HUANG, Shanghai (CN)

(73) Assignee: LifeArc, London, Greater London (GB)

(21) Appl. No.: 16/306,496

(22) PCT Filed: Jun. 2, 2017
(86) PCT No.: PCT/EP2017/063535

§ 371 Date: Nov. 30, 2018

(30) Foreign Application Priority Data
Jun. 3, 2016 (GB) 1609742.0

(51) Int. Cl. C07K 16/10; A61K 39/395; A61K 39/42; G01N 33/569; A61P 31/14

(52) U.S. Cl. CPC C07K 16/1009; A61K 39/39591; A61K 2039/505; G01N 33/56983; A61P 31/14; A61K 39/42

(57) ABSTRACT

The present invention provides humanized antibodies against Enterovirus 71 (EV71), a causative agent of hand, foot, and mouth disease (HFMD), as well as pharmaceutical compositions comprising these antibodies and methods of their use in the treatment, prophylaxis, and diagnosis of HFMD. These antibodies are suitable for use in human subjects with HFMD or those at risk of HFMD, for example as a result of exposure to infected individuals.

### RELATED APPLICATIONS

This application is the U.S. National Stage of International Application No. PCT/EP2017/063535, filed Jun. 2, 2017, which designates the U.S., published in English, and claims priority under 35 U.S.C. § 119 or 365(c) to Great Britain Application No. 1609742.0, filed Jun. 3, 2016. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file:
a) File name: 5630_1000_001_Corrected_Seq_Listing.txt; created Jan. 10, 2020, 29 KB in size.

### FIELD OF THE INVENTION

The present invention relates to humanized antibodies against Enterovirus 71 (EV71), a causative agent of hand, foot, and mouth disease (HFMD).

### BACKGROUND TO THE INVENTION

### HFMD

Hand, foot, and mouth disease (HFMD) is an infectious disease which frequently occurs in young children. Recently, HFMD infection has been prevalent in Asia-Pacific regions, causing severe illness and fatalities. For example, in 2012, China reported a total of 2,198,442 HFMD cases; among them, there are more than 20,000 severe cases with neurological complications, and 569 deaths.

Currently, no specific vaccine or antiviral drug is available for HFMD. Therefore, there is clearly an unmet medical need for treatments to combat this highly infectious disease.

### EV71

Enterovirus 71 (EV71) is one of the two main causative agents of HFMD (the other being Coxsackievirus A16 (CA16)). EV71 was initially identified as one of the causative pathogens for HFMD following isolation from the stool of a child aged 9 months with encephalitis in the USA in 1969. Over the next 5 years small outbreaks of neurological infections including encephalitis and aseptic meningitis across Australia, Japan, Sweden and the USA were reported and attributed to EV71 infection (Solomon T et al., Lancet Infect Dis, 2010 Nov;10(11):778-90). In the 1970's two large outbreaks of HFMD were reported in Europe, which were attributed to EV71 infection. Since 1997, EV71 has been shown to be responsible for outbreaks in the Asia-Pacific region with the largest Asia-Pacific epidemic reported in China in 2008. Brainstem encephalitis associated with cardiopulmonary dysfunction has become a notable feature in EV71-associated epidemics in Asia and is the primary cause of death.

A recent clinical survey indicated that 50.4% and 38.3% of the 266 laboratory confirmed HFMD cases was caused by EV71 and CA16 infection, respectively, during the 2009 HFMD outbreak in China. Individuals infected with EV71 usually present with mild symptoms, including fever, oral ulcers, and rashes on the surface of hands, feet and buttocks; however, a portion of the patients develop severe neurological complications, such as polio-like paralysis, brainstem encephalitis, and pulmonary edema, which may ultimately result in death. For example, a recent report shows that, out of 92 severe HFMD cases with neurological complications, 65 were infected with EV71.

EV71 is a member of the Enterovirus genus of Picornaviridae. It has a single-stranded, positive-sense RNA genome of ˜7.4 kb, which is packaged in an unenveloped icosahedral capsid. Antibodies against EV71 capsids can prevent the entry of virus into permissive cells, and therefore neutralize virus infection. Antibody-dependent cell-mediated cytotoxicity (ADCC) may also play a role in in vivo protection.

### Antibody Therapy

Vaccination against HFMD is a potential means to prevent or alleviate infection by EV71 or other causative agents of this disease. However, even where a vaccine against an infectious agent exists, this may not be an optimal route to reduce mortality caused by the agent. For example, it may not be economically viable to vaccinate a population, or it may be the case that uptake is lower than required to prevent outbreaks. For viruses such as EV71, an alternative option is to provide an antibody that can treat those infected individuals who develop complications, and/or provide preventative treatment to those in contact with such individuals.

### Antibodies Against EV71

US 2006/0292693 describes a monoclonal antibody derived from a mouse hybridoma cell line specific for neutralizing EV71 infection. The inventors of US 2006/0292693 propose that the antibody may be administered in a variety of formats, including as a humanized antibody. CN 103421112 A describes a different murine monoclonal anti-EV71 antibody, D5.

### Humanized Antibodies

It is generally desirable that murine antibodies developed for use in human subjects are humanized. In principle, this involves transferring the three complementarity determining regions (CDRs) of each of the murine heavy and light chain variable regions into the variable regions of a human antibody with framework regions that have a degree of homology to the murine framework regions. There are however a number of difficulties associated with humanizing antibodies. Simply transferring the CDRs into a human framework usually leads to a substantial loss of binding affinity compared to the original murine antibody. Furthermore, the humanized antibody may be more difficult to produce, and/or may have one or more undesirable physical or chemical properties, including the propensity to aggregate or a lack of thermal stability. Thus, even where a murine antibody is considered to be a candidate therapeutic agent, it may not be possible to provide a humanized version that will have a combination of binding affinity with other desirable physical and chemical properties that make it suitable to use in the clinic.

### DISCLOSURE OF THE INVENTION

The present inventors have investigated the D5 antibody. It has been determined that this antibody offers protection against lethal challenge of EV71 in new-born mice. In addition, treatment with D5 therapeutically at 1 day post-EV71 infection has also been shown to significantly improve the survival of EV71 infected animals.

It has also been found that D5 can inhibit EV71 binding to permissive cells and that D5 can neutralize EV71 at a post-attachment step. These properties indicate that D5 is an effective agent against EV71 infection and so can be used to treat HFMD in individuals infected with the EV71 strain.

It is an object of the invention to provide a humanized D5 antibody suitable for use in human subjects. Such subjects may be those with HFMD or those at risk of HFMD, e.g., through exposure to infected individuals. The present inventors identified heavy and light chain human framework regions that had primary sequences that were potentially suitable carriers for the CDRs of the D5 antibody. However, simply transferring the D5 CDRs into an apparently compatible human framework resulted in a humanized antibody with substantially lower binding to its target antigen. Following a detailed structural and functional analysis of the antibody, the present inventors were able to produce a modified antibody that has improved neutralization activity, as well as other physical and chemical properties that are desirable or necessary in clinical reagents.

In one aspect, the invention provides a humanized D5 antibody comprising a heavy chain variable domain and a light chain variable domain, wherein
a) the heavy chain variable domain (VH domain) comprises SEQ ID NO:1 having at least 6 of the following 8 substitutions: Y27X₆; T28X₂; F29X₁; T30X₄; R67X₄; V68X₁; R72X₁ and 598X₂; optionally with up to four additional framework substitutions, wherein each X₁ residue is independently selected from the group consisting of A, I, L, M, and V; each X₂ residue is independently selected from the group consisting of N, C, Q, S, and T; each X₄ residue is independently selected from the group consisting of R, H, and K; and the X₆ residue is selected from the group consisting of F and W; and
b) the light chain variable domain (VK domain) comprises SEQ ID NO:2; optionally with up to four framework substitutions.

The term “substitution(s)” above and herein below means that the amino acid residue “Xₙ” (where “n” is any of 1-6) is different from the amino acid present at the corresponding position of SEQ ID NO:1.

Specific examples of antibodies of the invention are described herein below. Where in the present description mention is made of products comprising, compositions of, or processes, methods or uses relating to, antibodies of the invention, such mention will be understood to refer to any antibody of the invention within the definition above, including but not limited to any specific examples of such antibodies mentioned herein below.

The invention further provides a nucleic acid encoding an antibody of the invention. The invention further provides a vector comprising the nucleic acid operably linked to a promoter. The invention further provides a host cell comprising such nucleic acids or vectors.

Also provided by the present invention are methods for making antibodies of the invention by expressing, in a host cell culture, a nucleic acid or vector of the invention to produce an antibody; and recovering the antibody from the cell culture.

The invention also provides a pharmaceutical composition comprising an antibody of the invention with a pharmaceutically acceptable carrier.

The invention also provides a method of treatment or prophylaxis of HFMD by administering, to an individual in need of treatment, an effective amount of an antibody or pharmaceutical composition of the invention. The individual may be with HFMD or one at risk of contracting HFMD.

The invention further provides an antibody, or pharmaceutical composition thereof, of the invention for use in a method of treatment of the human or animal body. The method of treatment may be the therapeutic or prophylactic treatment of HFMD in an individual.

The invention also provides diagnostic kits comprising an antibody of the invention.

### DESCRIPTION OF THE DRAWINGS

FIG. 1 depicts the sequences of the variable domains of the humanized heavy chains. The sequences of the native murine D5 VH and the human heavy chain donor candidate EF178053 are aligned. Humanized heavy chain variants D5 HA to D5 HQ are shown with highlighted residues indicating backmutations from the human residue in EF178053 to the corresponding murine D5 residue.

FIGS. 2a and 2b show that a chimeric D5 antibody (cD5) binds to EV71 VLP (virus-like particle) and the SP70 peptide epitope with comparable EC₅₀ values as those observed with the murine D5 (mD5) antibody. Binding was measured by ELISA over a concentration series.

[26] FIG. 3 shows that murine D5 (mD5) and chimeric D5 (cD5/chD5) antibodies bind to EV71-VLP with comparable apparent K_{D} values, thus demonstrating that they have a similar affinity for the virus. The concentration series was performed using Bio-Layer Interferometry.

FIG. 4 depicts the sequences of the variable domains of the light chain. Differences can be observed between the native murine D5 VK light chain and the human light chain donor candidate AJ388646. Humanized light chain variants D5 KA to D5 KE are depicted, with highlighted residues indicating the positions at which the human residue is backmutated to the equivalent murine D5 residue.

FIG. 5 displays the reduced binding capacity of humanized D5 antibody variants hEV71 HAKA, hEV71 HAKB, hEV71 HBKA and hEV71 HBKB to the EV71-VLP and the SP70 peptide epitope, when compared to chimeric D5 antibody (cD5 CHCK) and measured by binding ELISA.

FIG. 6 shows the reduced binding capacity of further humanized D5 antibody variants hEV71 HCKA to hEV71 HJKA to the SP70 peptide epitope, when compared to chimeric D5 antibody (cD5 CHCK) and measured by binding ELISA.

FIG. 7 shows the EV71 virus neutralization capability of the humanized D5 antibody variants hEV71 HAKA to hEV71 HJKA and hEV71 HAKB to hEV71 HJKB. The only antibody variants able to neutralize the virus were those comprising the HB heavy chain variant. cD5 is the chimeric D5 antibody, mD5 IgG1 is the native murine D5 antibody.

FIG. 8 shows the binding capacity of humanized D5 antibody variants containing the HB, HM, HN, HP and HQ heavy chain variants, in combination with the KA light chain, to the SP70 peptide epitope. Binding was measured by binding ELISA. hEV71 HBKA and hEV71 HNKA display a similar binding capacity. hEV71 HMKA and hEV71 HQKA display a greater binding capacity for the SP70 epitope, similar to that of chimeric D5 antibody (cD5 CHCK). hEV71 HPKA shows poor binding to the SP70 epitope.

FIG. 9 depicts the binding ability of the hEV71 HBKA, HMKA, HNKA, HPKA, and HQKA antibody variants to 15 variations of the SP70 peptide epitope. Binding was measured by binding ELISA at the EC₈₀ of each antibody. The SP70 epitope variations were created by alanine scanning. The hEV71 variants comprising the HM, HN, HP and HQ heavy chains demonstrate the same pattern of binding to the SP70 epitope library as the chimeric D5 antibody (cEV71 CHCK) and hEV71 HBKA variant.

FIG. 10 displays the EV71 virus neutralization capacity of the hEV71 HMKA to hEV71 HQKA antibody variants. hEV71 HNKA shows a small improvement on virus neutralization compared to hEV71 HBKA, and hEV71 HPKA does not neutralize the virus. hEV71 HMKA and hEV71 HQKA show a significant improvement over the HBKA variant and a smaller improvement over the murine D5 antibody (mD5 IgG1).

FIG. 11 shows the binding of chimeric D5 antibody (cD5) and hEV71 HMKA to the SP70 peptide epitope, performed by isothermal titration calorimetry. The observed binding constant (K_{A}), observed dissociation constant (K_{D}), enthalpy (ΔH) and entropy (ΔS) were calculated for both antibodies. Chimeric D5 antibody and hEV71 HMKA have similar K_{A} and K_{D} values, indicating that they bind the SP70 epitope with similar affinity.

FIG. 12 illustrates the thermal stability of hEV71 HMKA antibody. FIG. 12a displays the binding activity of the antibody to the EV71 SP70 epitope after temperature treatments between 25° C. and 80° C., measured by binding ELISA. hEV71 HMKA retained epitope binding activity until 70° C. FIG. 12b shows the melting temperature (Tm) is approximately 70° C. in a thermal shift assay.

FIG. 13 shows that hEV71 HMKA does not have a propensity to aggregate. The antibody was injected at 0.4 ml/min into a size exclusion column in an HPLC system and analysed by multi-angle light scattering to determine the absolute molar masses and check for aggregation. The profile shows no signs of aggregation, and the average molecular weight of approximately 136.6 kDa is expected for an IgG monomer. A polydispersity value of 1.00 reveals that the antibody is monodispersed, and the high mass recovery indicates that the antibody did not stick to the column or contain insoluble aggregates.

FIG. 14 shows that hEV71 HMKA has a low propensity for non-specific interactions and a good solubility. Cross-Interaction Chromatography was performed to assess non-specific interactions and solubility. The low Retention Index (k′) of hEV71 HMKA shows that the antibody is less likely to be involved with non-specific interactions and has good solubility.

FIG. 15 shows that the hEV71 HMKA antibody can be concentrated up to ˜100 mg/ml without apparent precipitation. Dynamic light scattering (DLS) was used to test for soluble aggregates. The mean particle size (Z-Ave diameter) and the polydispersity index (PDI) were obtained by cumulants analysis and show similar values for the pre-concentrated and post-concentrated antibody samples.

FIG. 16 shows that subjecting hEV71 HMKA to freeze/thaw treatment does not cause aggregation. Samples of purified hEV71 HMKA were treated with 10 cycles of 15 minutes at −80° C. followed by thawing for 15 minutes at room temperature and analysed by SEC-MALS (size exclusion chromatography—multi angle light scattering). Treated hEV71 HMKA samples contained species with a molecular weight in the range of monomeric IgG and the antibody was monodispersed (i.e., one molecular weight is present).

FIG. 17 depicts the SEC-MALS analysis of hEV71 HMKA antibody exposed to 4° C., 25° C., 37° C. or 50° C. for 33 days. No aggregation of the antibody is apparent, the molecular weight is similar to that of monomeric IgG, and the antibody is monodispersed.

FIG. 18 shows that the hEV71 antibody is stable for one month in mouse, human and cynomolgus serum. The antibody was incubated in each serum, or a PBS control, for 30 days, and binding of the antibody to the SP70 peptide epitope after the incubation period was measured by binding ELISA. The binding of the serum incubated antibody to the SP70 peptide is very similar to the binding of the PBS incubated and non-incubated antibody.

FIG. 19 shows that treatment with hEV71 HMKA results in 100% survival after 14 days of mice challenged with EV71 virus.

FIG. 20 shows the results of hEV71 HM KA antibody buffer scout by measurement of size distribution in 10, 25 and 50 mM sodium acetate (pH 5.2, 130 mM NaCl) buffers.

FIG. 21 shows the results of hEV71 HM KA antibody buffer scout by measurement of size distribution in 10, 25 and 50 mM sodium phosphate (pH 7.0, 135 mM NaCl) buffers.

FIG. 22 shows the results of hEV71 HM KA antibody buffer scout by measurement of size distribution in 10, 25 and 50 mM sodium succinate (pH 6.0, 125 mM NaCl) buffers.

FIG. 23 shows the results of hEV71 HM KA antibody buffer scout by measurement of size distribution in 10, 25 and 50 mM sodium citrate (pH 6.0 125 mM NaCl) buffers.

FIG. 24 shows that treatment with antibody hEV71 HM KA results in a reduced increase in body temperature following infection with EV71 in a rhesus monkey model (treatment group compared with the model control group: *p<0.05, **p<0.01; treatment group compared with the control antibody group: #p<0.05, #4p<0.01).

FIG. 25 shows that treatment with antibody hEV71 HM KA results in a reduced post-infection viral load 3-4 days after infection in a rhesus monkey model (treatment group compared with the model control group, *p<0.05, **p<0.01; treatment group compared with the control antibody group, #p<0.05, ##p<0.01).

### DETAILED DESCRIPTION OF THE INVENTION

### Antibodies of the Invention

An antibody of the invention comprises a heavy chain variable domain and a light chain variable domain of SEQ ID NO:1 and SEQ ID NO:2, respectively, modified as defined above. In the present specification, where residue numbering is defined in relation to sequence identifiers (i.e., of the format SEQ ID NO:n), the residues are numbered consecutively from the first residue. In the accompanying examples and drawings, the numbering of amino acids is also discussed in accordance with the definitions of Kabat, Sequences of Proteins of Immunological Interest (National Institutes of Health, Bethesda, Md., 1987 and 1991). Where Kabat numbering is used, this is indicated or will be clear from the context.

The inventors have identified a number of framework residues of SEQ ID NO:1 and SEQ ID NO:2 that may be altered to modify the properties of an antibody comprising a heavy chain variable domain of SEQ ID NO:1 and a light chain variable domain of SEQ ID NO:2. In the accompanying examples, the framework residues were altered to revert to those of the corresponding murine framework sequence. However, any of the amino acid residues which are substitutes for VH and VK domain human framework residues, for example the Thr residue in the K23T substitution as described above, can be themselves substituted for another amino acid with similar properties, i.e. a conservative change to an amino acid in the same functional group. For example, the K23T substitution can be a K23S substitution as Thr and Ser both have polar uncharged side chains.

For conservative residue substitutions, amino acids can be grouped as follows: Group X₁ (non-polar side chains): Ala (A), Ile (I), Leu (L), Met (M), Val (V); Group X₂ (polar uncharged side chains): Asn (N), Cys (C), Gin (Q), Ser (S), Thr (T); Group X₃ (acidic side chains): Asp (D), Glu (E); Group X₄ (basic side chains): Arg (R), His (H), Lys (K); Group X₅ (residues influencing chain orientation with non-polar side chains): Gly (G), Pro (P); and Group X₆ (aromatic non-polar side chains): Phe (F), Trp (W), Tyr (Y).

Accordingly, heavy chain variable domains (VH domains) of the present invention comprise SEQ ID NO:1 having at least 6, or at least 7, of the following 8 substitutions: Y27X₆; T28X₂; F29X₁; T30X₄; R67X₄; V68X₁; R72X₁ and 598X₂; optionally with up to four additional framework substitutions. A heavy chain variable domain containing all 8 of this set of substitutions is designated in Table 1A below as the HB′ chain.

Preferably, the at least 6 substitutions include 598X₂. When 598X₂ is present, it is preferably S98N.

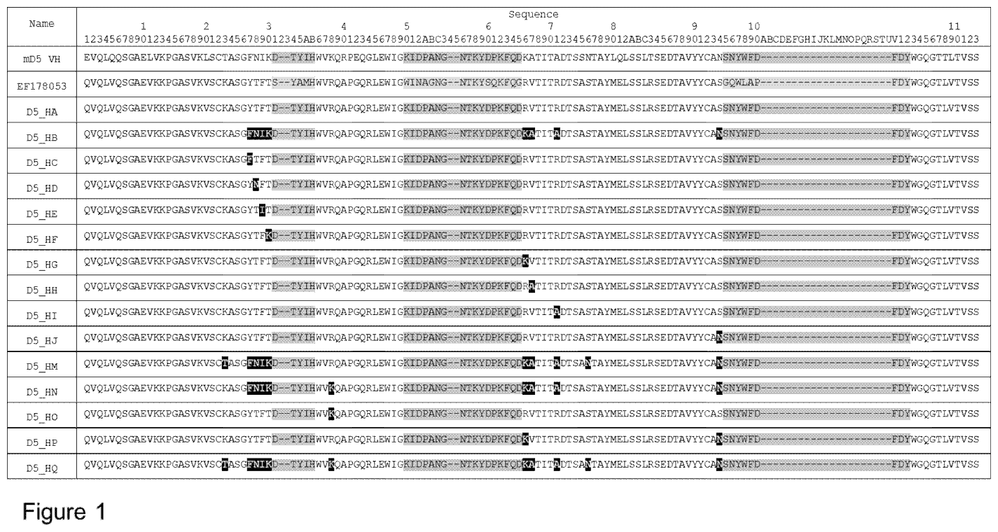

In one aspect, the at least 6, or at least 7, or all 8 substitutions are selected from the group of Y27F; T28N; F29I; T30K; R67K; V68A; R72A and S98N. Preferably, the at least 6 substitutions include S98N. This group of 8 substitutions are collectively referred to as the HB domain group substitutions. A heavy chain variable domain containing all 8 specific substitutions as described above is designated the HB chain (SEQ ID NO:11).

Where up to four additional framework substitutions may be present, this may be 1, 2, 3 or 4 additional substitutions. The substitutions are preferably at residues of SEQ ID NO:1 that differ from the corresponding residue of the murine heavy chain variable domain. The substituted residues are indicated in the alignment shown in FIG. 1. Three of the four substitutions may be selected from K23X₂, R38X₄ and 577X₂, wherein each X₂ residue is independently selected from the group consisting of N, C, Q, S, and T, and the X₄ residue is selected from the group consisting of R, H, and K. Generally back-substitutions to those of corresponding residue of the murine antibody sequence may be made. Three of the four substitutions may be selected from K23T, R38K and S77N.

The framework residues of SEQ ID NO:1 are residues 1-30, 36-49, 67-98 and 108-118.

In the accompanying examples, SEQ ID NO:1 is referred to as the HA domain. Examples of VH domains of the invention comprise the sequence of the HA chain substituted at 6, 7 or all 8 of the HB′ domain group substitutions include those combinations set out in Table 1A:

TABLE 1A

| Domain | Y27X₆ | T28X₂ | F29X₁ | T30X₄ | R67X₄ | V68X₁ | R72X₁ | S98X₂ |
|---|---|---|---|---|---|---|---|---|
| Hb'1 | · | · | · | · | · | · | | |
| Hb'2 | · | · | · | · | · | | · | |
| Hb'3 | · | · | · | · | · | | | · |
| Hb'4 | · | · | · | · | | · | · | |
| Hb'5 | · | · | · | · | | · | | · |
| Hb'6 | · | · | | · | · | · | | · |
| Hb'7 | · | · | · | | · | · | | · |
| Hb'8 | · | · | | | · | · | · | · |
| Hb'9 | · | · | · | · | · | · | · | |
| Hb'10 | · | · | · | · | · | · | | · |
| HB' | · | · | · | · | · | · | · | · |

where “·” indicates that the HA domain of SEQ ID NO: 1 has been substituted as indicated in the header row of the Table.

Where six, seven or all eight of the HB′ group substitutions are present, these may be combined with (a) K23X₂, R38X₄ & S77X₂; (b) K23X₂ & R38X₄; (c) R38X₄ & S77X₂; (d) K23X₂ & S77X₂; (e) K23X₂; (f) R38X₄; or (g) S77X₂; wherein each X₂ residue is independently selected from the group consisting of N, C, Q, S, and T, and the X₄ residue is selected from the group consisting of R, H, and K. A heavy chain comprising any combination of these substitutions can be present in an antibody in combination with any of the light chain sequences described herein.

For example, Hb′1 may be combined with any of the combinations of further substitutions set out as (a), (b), (c), (d), (e), (f) or (g) above; Hb′2 may be combined with any of the combinations of further substitutions set out as (a), (b), (c), (d), (e), (f) or (g) above; Hb′3 may be combined with any of the combinations of further substitutions set out as (a), (b), (c), (d), (e), (f) or (g) above; Hb′4 may be combined with any of the combinations of further substitutions set out as (a), (b), (c), (d), (e), (f) or (g) above Hb′5 may be combined with any of the combinations of further substitutions set out as (a), (b), (c), (d), (e), (f) or (g) above; Hb′6 may be combined with any of the combinations of further substitutions set out as (a), (b), (c), (d), (e), (f) or (g) above; Hb′7 may be combined with any of the combinations of further substitutions set out as (a), (b), (c), (d), (e), (f) or (g) above Hb′8 may be combined with any of the combinations of further substitutions set out as (a), (b), (c), (d), (e), (f) or (g) above; Hb′9 may be combined with any of the combinations of further substitutions set out as (a), (b), (c), (d), (e), (f) or (g) above; Hb′10 may be combined with any of the combinations of further substitutions set out as (a), (b), (c), (d), (e), (f) or (g) above HB′ may be combined with any of the combinations of further substitutions set out as (a), (b), (c), (d), (e), (f) or (g) above.

In a preferred embodiment, VH domains of the invention comprise the sequence of the HA chain substituted at 6, 7 or all 8 of the HB domain group substitutions include those combinations set out in Table 1B:

TABLE 1B

| Domain | Y27F | T28N | F29I | T30K | R67K | V68A | R72A | S98N |
|---|---|---|---|---|---|---|---|---|
| Hb1 | · | · | · | · | · | · | | |
| Hb2 | · | · | · | · | · | | · | |
| Hb3 | · | · | · | · | · | | | · |
| Hb4 | · | · | · | · | | · | · | |
| Hb5 | · | · | · | · | | · | | · |
| Hb6 | · | · | | · | · | · | | · |
| Hb7 | · | · | · | | · | · | | · |
| Hb8 | · | · | | | · | · | · | · |
| Hb9 | · | · | · | · | · | · | · | |
| Hb10 | · | · | · | · | · | · | | · |
| HB | · | · | · | · | · | · | · | · |

where “·” indicates that the HA domain of SEQ ID NO: 1 has been substituted as indicated in the header row of the Table.

Where six, seven or all eight of the HB group substitutions are present, these may be combined with (i) K23T, R38K & S77N; (ii) K23T & R38K; (iii) R38K & S77N; (iv) K23T & S77N; (v) K23T; (vi) R38K; or (vii) S77N. A heavy chain comprising any combination of these substitutions can be present in an antibody in combination with any of the light chain sequences described herein.

For example, Hb1 may be combined with any of the combinations of further substitutions set out as (i), (ii), (iii), (iv), (v), (vi) or (vii); Hb2 may be combined with any of the combinations of further substitutions set out as (i), (ii), (iii), (iv), (v), (vi) or (vii); Hb3 may be combined with any of the combinations of further substitutions set out as (i), (ii), (iii), (iv), (v), (vi) or (vii); Hb4 may be combined with any of the combinations of further substitutions set out as (i), (ii), (iii), (iv), (v), (vi) or (vii); Hb5 may be combined with any of the combinations of further substitutions set out as (i), (ii), (iii), (iv), (v), (vi) or (vii); Hb6 may be combined with any of the combinations of further substitutions set out as (i), (ii), (iii), (iv), (v), (vi) or (vii); Hb7 may be combined with any of the combinations of further substitutions set out as (i), (ii), (iii), (iv), (v), (vi) or (vii); Hb8 may be combined with any of the combinations of further substitutions set out as (i), (ii), (iii), (iv), (v), (vi) or (vii); Hb9 may be combined with any of the combinations of further substitutions set out as (i), (ii), (iii), (iv), (v), (vi) or (vii); Hb10 may be combined with any of the combinations of further substitutions set out as (i), (ii), (iii), (iv), (v), (vi) or (vii); HB may be combined with any of the combinations of further substitutions set out as (i), (ii), (iii), (iv), (v), (vi) or (vii).

Examples of VH domains of the invention include the HM (SEQ ID NO:3), HN (SEQ ID NO:4) and HQ (SEQ ID NO:6) heavy chains set out in the accompanying examples.

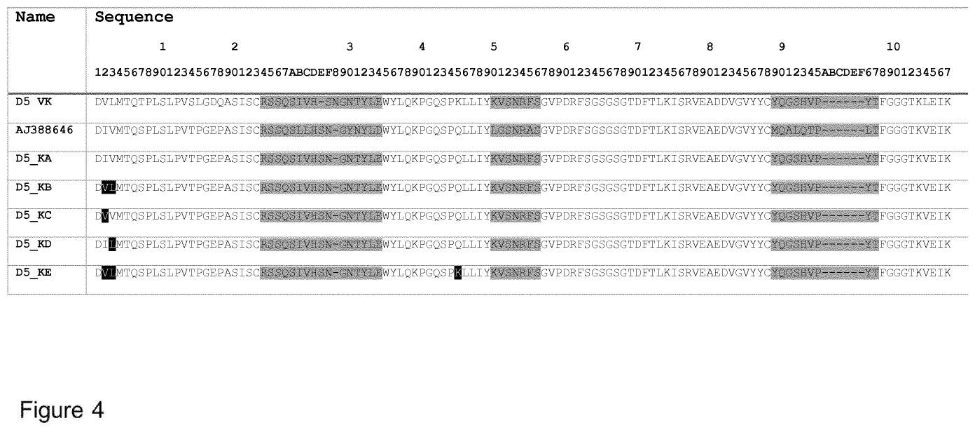

Light chain variable domains (VK domains) of the present invention comprise SEQ ID NO:2; optionally with up to four framework substitutions, such as 3, 2 or 1 substitutions. SEQ ID NO:2 is referred to herein as the KA domain. The substitutions are preferably at residues of SEQ ID NO:2 that differ from the corresponding residue of the murine heavy chain variable domain. The substituted residues are indicated in the alignment shown in FIG. 4. The substitutions may include 1, 2, or 3 of I2X₁, V3X₁, and Q50X₄, wherein each X₁ residue is independently selected from the group consisting of A, I, L, M, and V; and the X₄ residue is selected from the group consisting of R, H, and K. The term “substitution” means that the amino acid residue “X₁” is different from the amino acid present at the corresponding position of SEQ ID NO:2. A light chain comprising any combination of these substitutions can be present in an antibody in combination with any of the heavy chain sequences described herein above.

The light chain domain substitutions are generally back-substitutions to those of corresponding residue of the murine antibody sequence. Substitutions of KA may include 1, 2 or 3 of I2V, V3L, and Q50K. Examples of substituted KA domains include the variable domains designated KB (SEQ ID NO:7), KC (SEQ ID NO:8), KD (SEQ ID NO:9) and KE (SEQ ID NO:10).

The framework residues of SEQ ID NO:2 are 1-23, 40-54, 62-93 and 103-112.

Antibodies of the present invention may comprise any of the above-mentioned heavy VH domains in combination with each and any of the above-mentioned VK domains. In one embodiment, the KA domain is preferred, and this may be used in combination with each and any of the VH domains mentioned above.

In one embodiment, an antibody of the present invention comprises the VH domain of SEQ ID NO:3 and the VK domain of SEQ ID NO:2. This antibody is referred to in the examples as hEV71 HMKA.

For the avoidance of doubt, the terms “having at least 6 of the following 8 substitutions” and “optionally with up to four additional framework substitutions” read in combination indicate that a heavy chain variable domain of the invention has the sequence of SEQ ID NO:1 apart from substitutions of at least 6, and up to 12 in total, framework residues, with the remainder of the sequence being that as set out in SEQ ID NO:1. Where specific combinations of 1, 2 or 3 additional framework substitutions are mentioned, heavy chain variable domain will have a total of at least 6 of the 8 Y27X₆; T28X₂; F29X₁; T30X₄; R67X₄; V68X₁; R72X₁ and 598X₂ together with the additional specific number of additional framework substitutions; e.g., at total of 7-9, 8-10 or 9-11, respectively, framework substitutions. Similarly, a light chain variable domain of the invention may have in total 4 framework substitutions of SEQ ID NO:2, such as a total of 0, 1, 2 3 or 4, with the remainder of the sequence being that of SEQ ID NO:2.

### Antibody Structure

The term “immunoglobulin” or “antibody” may be used interchangeably to refer to a protein which has the ability to specifically bind one or more antigens. Native antibodies are usually heterotetrameric glycoproteins of about 150,000 Daltons, composed of two identical light (L) chains and two identical heavy (H) chains. Each light chain is linked to a heavy chain by one covalent disulphide bond, with varying numbers of disulphide linkages between the heavy chains of different antibody isotypes. Each heavy and light chain also has regularly spaced intrachain disulphide bridges.

An antibody comprises globular regions of heavy or light chain polypeptides called “domains”. A domain comprises peptide loops, usually 3 to 4 loops, which are stabilized, for example, by β-pleated sheet and/or intrachain disulphide bonding. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”.

The “constant” domains of an antibody light chain may be referred to as “light chain constant regions”, “light chain constant domains”, “CL” regions or “CL” domains. The “constant” domains of an antibody heavy chain may be referred to as “heavy chain constant regions”, “heavy chain constant domains”, “CH” regions or “CH” domains. The constant domain of the light chain is aligned with the first constant domain of the heavy chain

The constant domain of the heavy chain which comprises the tail region of the antibody is referred to herein as the Fc (fragment crystallizable) domain or Fc region. The Fc region can interact with cell surface Fc receptors and some proteins of the complement system, by which method the antibody can activate the immune system. The Fc regions contain three heavy chain constant domains in each polypeptide chain.

The “variable” domains of an antibody light chain may be referred to as “light chain variable regions”, “light chain variable domains”, “VL” regions or “VL” domains (the ‘L’ here referring to ‘light’ rather than the light chain isotype ‘lambda’). The “variable” domains of an antibody heavy chain may be referred to as “heavy chain variable regions”, “heavy chain variable domains”, “VH” regions or “VH” domains.

Intact light chains have, for example, two domains (VL and CL) and intact heavy chains have, for example, four or five domains (VH, CH1, CH2, and CH3).

Light and heavy chain variable domains include “hypervariable regions” (HVR or HV), also known as “complementarity determining regions” or “CDRs”, which are hypervariable in sequence and may form structurally defined loops. Generally, antibodies comprise six hypervariable regions; three in the heavy chain (H1, H2, H3) and three in the light chain (L1, L2, L3) interspersed among relatively conserved framework regions (FRs). In antibodies of the present invention, the amino acid sequences of the variable domains and CDRs are as defined herein, with reference to FIG. 1, FIG. 4, and the sequence listing.

The variable regions of each light/heavy chain pair form the antigen binding site. The term “antigen binding site” refers to a site that specifically binds (immunoreacts with) an antigen. Antibodies of the invention comprise at least one antigen binding site, preferably comprising two antigen binding sites. An antigen binding site is formed from the heavy and light chain CDRs, aligned by the framework regions, which enable binding to a specific epitope. An “antigen binding region” or “antigen binding domain” is an antibody region or domain that includes an antibody binding site. Antibodies of the present invention have at least one antigen binding site which can recognize the SP70 epitope of EV71.

Naturally-occurring antibody chains or recombinantly-produced antibody chains can be expressed with a leader sequence which is removed during cellular processing to produce a mature chain. Mature chains can also be produced recombinantly, containing a non-naturally occurring leader sequence, for example, to enhance secretion or alter the processing of a particular chain of interest.

### Antibody Isotypes

The constant regions of the heavy and light chains that comprise an antibody display phenotypic variation. Antibody light chains are classified as either kappa (κ) or lambda (λ) based on the amino acid sequence of the light chain constant region, and are about 230 residues in length. An antibody of the present invention comprises a kappa light chain (the variable domain of the kappa light chain is referred to herein as VK).

Heavy chains from humans and higher mammals are classified as gamma (γ), mu (μ), alpha (α), delta (δ), or epsilon (ε), are about 450-600 residues in length, and define the antibody's isotype as IgG, IgM, IgA, IgD and IgE, respectively. There are two subclasses of IgM (H and L), three subclasses of IgA (IgA1, IgA2, and secretory IgA), and four subclasses of IgG (IgG1, IgG2, IgG3, and IgG4). An antibody of the present invention is preferably an immunoglobulin G (IgG) antibody. An antibody of the present invention is more preferably an IgG1 antibody.

The antibodies of the present invention may comprise heavy chains which belong to any of the immunoglobulin isotypes described herein. The antibodies of the present invention may comprise sequences from more than one class or isotype.

An antibody of the present invention may exhibit cytotoxic activity. In such an antibody, the constant domain is usually a complement fixing constant domain and the class is typically IgG1. Human isotypes IgG1 and IgG4 are exemplary.

### Antibody Formats

An antibody of the present invention may comprise a fragment of an antibody. The term “fragment” refers to a part or portion of an antibody or antibody chain comprising fewer amino acid residues than an intact or complete antibody or antibody chain, wherein the portion preferably retains at least one, preferably most or all, of the functions normally associated with that portion when present in an intact antibody. Fragments can be obtained via chemical or enzymatic treatment of an intact or complete antibody or antibody chain. Fragments can also be obtained by recombinant means.

Antibodies of the present invention may exist as fragments including, but not limited to, Fab, Fab′, F(ab′)₂, chemically linked F(ab′)₂, monospecific Fab₂, bispecific Fab₂, trispecific Fab₂, monovalent IgG, scFv (single-chain variable fragment), di-scFv (divalent scFv), bispecific diabody, trispecific triabody, scFv-Fc, minibody and sdAb (single domain antibody) fragments.

Fragments of the antibodies of the present invention can bind antigen or compete with intact antibody (i.e., with the intact antibody from which they were derived) for antigen binding (i.e., specific binding). Antibodies of the present invention bind to the SP70 peptide binding epitope on the EV71 VP1 capsid protein. Binding fragments are produced by recombinant DNA techniques, or by enzymatic or chemical cleavage of intact immunoglobulins. Antibodies of the present invention may exist as binding fragments including, but not limited to, Fab, Fab′, F(ab′)₂, chemically linked F(ab′)₂, monospecific Fab₂, bispecific Fab₂, trispecific Fab₂, monovalent IgG, scFv (single-chain variable fragment), di-scFv (divalent scFv), bispecific diabody, trispecific triabody, scFv-Fc, minibody or sdAb (single domain antibody), and retain the ability to bind the SP70 epitope of EV71.

An antibody of the present invention can be part of a bispecific or trispecific antibody. A bispecific is an artificial hybrid antibody having two different heavy/light chain pairs and two different antigen-binding sites; a trispecific antibody is an artificial hybrid antibody having three different heavy/light chain pairs and three different antigen-binding sites. Bispecific and trispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

An exemplary antibody of the present invention can be a bispecific antibody comprising at least two different antigen binding sites. In a particular embodiment, one antigen binding site can bind to the SP70 peptide epitope of EV71, and one antigen binding site can bind to another infectious agent, e.g., an antigen binding site that can bind to the Coxsackievirus A16 (CA16) virus.

### Antibody Characteristics

An antibody of the present invention has structural features as described herein, and specifically binds to the SP70 binding epitope (SEQ ID NO:12) of the EV71 VP1 protein (see Example 4).

An antibody of the present invention can neutralize the EV71 virus (see Example 5).

An antibody of the present invention can also have desirable structural, physical, biophysical and chemical properties as described herein below, and with reference to the examples.

### Antibody Binding Affinity

The affinity of an antibody as described herein is the extent or strength of binding of antibody to epitope or antigen. The dissociation constant, K_{d}, and the affinity constant, Kₐ, are quantitative measures of affinity. K_{d} is the ratio of the antibody dissociation rate (k_{off}), how quickly it dissociates from its antigen, to the antibody association rate (kₒₙ) of the antibody, how quickly it binds to its antigen. The binding of an antibody to its antigen is a reversible process, and the rate of the binding reaction is proportional to the concentrations of the reactants. At equilibrium, the rate of [antibody][antigen] complex formation is equal to the rate of dissociation into its components [antibody]+[antigen]. The measurement of the reaction rate constants can be used to define an equilibrium or affinity constant, Kₐ (Kₐ=1/K_{d}). The smaller the K_{d} value, the greater the affinity of the antibody for its target. Most antibodies have K_{d} values in the low micromolar (10⁻⁶) to nanomolar (10⁻⁷ to 10⁻⁹) range. High affinity antibodies are generally considered to be in the low nanomolar range (10⁻⁹) with very high affinity antibodies being in the picomolar (10⁻¹²) range.

[92] An antibody of the present invention can have an association rate constant (Kₒₙ) of at least 2×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or least 10⁸ M⁻¹s⁻¹.

An antibody of the present invention can have an antibody dissociation (k_{off}) rate of less than 5×10⁻¹ s⁻¹, less than 10⁻¹ s⁻¹, less than 5×10⁻² s⁻¹, less than 10⁻² s⁻¹, less than 5×10⁻³ s⁻¹, less than 10⁻³ s⁻¹, less than 5×10⁻⁴ s⁻¹, or less than 10⁻⁴ s⁻¹. In a another embodiment, an antibody of the present invention can have a k_{off} of less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, or less than 10⁻¹ 0 s⁻¹.

In an embodiment, an antibody of the present invention binds (e.g. specifically binds) to the SP70 peptide binding epitope (SEQ ID NO:12) of EV71 with an affinity constant or Kₐ of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, or at least 10¹¹ M⁻¹.

An antibody of the present invention can have a dissociation constant or K_{d} from the SP70 epitope of EV71 of less than 5×10⁻² M, less than 10⁻² M, less than 5×10⁻³ M, less than 10⁻³ M, less than 5×10⁻⁴ M, less than 10⁻⁴ M, less than 5×10⁻⁵ M, less than 10⁻⁵ M, less than 5×10⁻⁶ M, less than 10⁻⁶ M, less than 5×10⁻⁷ M, less than 10⁻⁷M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, or less than 10⁻¹² M.

In a preferred embodiment, an antibody of the invention binds the SP70 epitope of EV71 with a Kₐ of between 10⁶ M⁻¹ and 10⁸ M⁻¹. In a further example, an antibody of the invention binds to the SP70 epitope of EV71 with a Kₐ of between 10⁷ M⁻¹ and 5×10⁷ M⁻¹. In a further example still, an antibody of the invention binds to the SP70 epitope of EV71 with a Kₐ of between 1.5×10⁷ M⁻¹ and 3.5×10⁷ M⁻¹ (see Example 6).

In a preferred embodiment, an antibody of the invention has a K_{d} of between 10⁻⁶ M and 10⁻⁹ M. In a further example, an antibody of the invention has a K_{d} of between 5×10⁻⁷M and 5×10⁻⁹ M. In a further example still, an antibody of the invention has a K_{d} of between 10⁻⁷M and 10⁻⁸ M (see Example 6).

Specific binding of an antibody means that the antibody exhibits appreciable affinity for a particular antigen or epitope and, generally, does not exhibit significant crossreactivity. An antibody that “does not exhibit significant crossreactivity” is one that will not appreciably bind to an undesirable entity (e.g., an undesirable proteinaceous entity). An antibody specific for a particular epitope will, for example, not significantly crossreact with remote epitopes on the same protein or peptide. Specific binding i.e., k_{off}, kₒₙ, Kₐ and K_{d}, of an antibody of the present invention can be determined according to any art-recognized means for determining such binding.

### Biophysical Properties

An antibody of the present invention can be thermally stable, i.e., an antibody of the present invention can bind to the SP70 binding epitope (SEQ ID NO:12) of the EV71 virus at temperatures between 30° C. and 85° C., specifically up to 75° C. (see Example 7). An antibody of the present invention can have a melting temperature of between 50° C. and 100° C., specifically between 60 and 80° C., more specifically near 70° C. (see Example 7).

An antibody of the present invention can have a low propensity for aggregation. In one example the propensity for aggregation is analysed using multi-angle light scattering, in another example the propensity for aggregation is analysed using dynamic light scattering. An antibody of the present invention can have a low propensity for non-specific protein-protein interactions and good solubility (see Example 8).

An antibody of the present invention can have a low propensity for aggregation when concentrated (see Example 8). A formulation of the present invention can comprise an antibody concentrated to 50-200 mg/ml, for example 75-150 mg/ml, preferably 80-120 mg/ml and more preferably 90-110 mg/ml, with a preferred concentration of about 100 mg/ml, without forming soluble aggregates in an aqueous solution maintained at physiological pH, for example by Dulbecco's PBS.

An antibody of the present invention can have a low propensity for aggregation when subjected to repeated freezing and thawing, or prolonged temperatures above normal body temperature. In an example, a prolonged temperature is 50° C. for 33 days in Dulbecco's PBS.

An antibody of the present invention can have an isoelectric point (pl) between pH 6.6 and pH 7.6. In an example, an antibody of the invention has a pl between pH 7 and pH 7.5. In an even further example, an antibody of the invention has a pl of pH 7.3 to pH 7.4, preferably having a pl of pH 7.4 (see Example 8).

An antibody of the present invention can retain binding capability to the SP70 peptide epitope (SEQ ID NO:12) of EV71 after incubation at 37° C. in serum from a mouse, human and/or cynomolgus primates (see Example 9). In an example, an antibody of the invention can retain binding capability to the SP70 peptide epitope after incubation in mouse, human and/or cynomolgus serum for 10 to 50 days, preferably 20-40 days, more preferably 30 days. In one example, retaining binding capability refers to the antibody displaying the same or substantially the same binding capability at 37° C. as that observed in an antibody which was not incubated in serum, or which was incubated in a control solution.

In an embodiment, an antibody of the present invention has structural features, as described herein, and further has at least one of the following activities or functional features: (i) binds EV71; (ii) binds the SP70 epitope of EV71; (iii) neutralizes EV71 virus infection, (iv) has a Kₐ value as described herein; (v) has a K_{d} value as described herein; (vi) retains SP70 epitope binding activity until 75° C.; (vii) has a melting temperature of 70° C.; (viii) does not aggregate when concentrated to 100 mg/ml; (ix) has a Retention Index (k′) below 0.037 when analysed by cross-interaction chromatography; (x) does not aggregate when subjected to repeated cycles of freezing and thawing, or when treated at up to 50° C. for up to 33 days; (xi) has an isoelectric point (p1) as described herein; (xii) retains the ability to bind to the SP70 epitope of EV71 when incubated in mouse, human or cynomolgus serum for up to 30 days.

### Aglycosylated Antibodies

An antibody disclosed herein can be an “aglycosylated” antibody. The Fc regions of IgG antibodies bear a highly-conserved N-glycosylation site and glycosylation of the Fc fragment is essential for Fc receptor-mediated activity. The N-glycan carbohydrate moieties attached to this site are predominantly core-fucosylated diantennary structures of the complex type. In addition, small amounts of these N-glycans also bear bisecting GlcNAc and α-2,6 linked sialic acid residues.

“Aglycosylated” refers to an antibody lacking one or more carbohydrate moieties by virtue of, but not limited to, a chemical or enzymatic process, mutation of one or more glycosylation sites or expression in bacteria. An aglycosylated antibody of the present invention can be a deglycosylated antibody, that is an antibody for which the Fc carbohydrate has been removed, for example, chemically or enzymatically. Alternatively, the aglycosylated antibody of the present invention can be a nonglycosylated or unglycosylated antibody, that is an antibody that was expressed without Fc carbohydrate moieties, for example by mutation of one or more residues that encode the glycosylation pattern or by expression in an organism that does not attach carbohydrates to proteins, for example bacteria.

Antibodies described herein may be “afucosylated”, i.e. engineered so that the carbohydrate moieties in the Fc region of the antibody do not have any fucose sugar units. Alternatively, an antibody described herein may have a reduced number of fucose sugar units. Afucosylated antibodies are more effective in antibody-dependent cell-mediated cytotoxicity (see below). Afucosylated antibodies described herein can be produced in cell lines engineered to produce afucosylated proteins, such as the Potelligent® CHOK1SV cell line (BioWa/Lonza), GlymaxX®-engineered cells (ProBioGen) or the duck embryonic stem cell line EB66 (Valneva).

### Antibody-Dependent Cell-Mediated Cytotoxicity

An antibody as described herein can be modified to enhance its antibody-dependent cell-mediated cytotoxicity (ADCC). ADCC is a cell-mediated reaction in which non-specific cytotoxic cells (e.g., Natural Killer (NK) cells, neutrophils, and macrophages) recognize bound antibody on a target cell and subsequently cause lysis of the target cell. Such a cell can be a human cell. ADCC activity of antibodies is generally thought to require the binding of the Fc region of an antibody to an antibody receptor existing on the surface of an effector cell, such as, for example, a killer cell, a natural killer cell and an activated macrophage. By altering fucosylation (e.g., reducing or eliminating) of the carbohydrate structure of a humanized antibody (i.e., in the Fc region), the ADCC activity of the antibody can be enhanced in vitro by, for example, 10-fold, or 20-fold, or 30-fold, or 40-fold, or 50-fold, or 100-fold, or 500-fold, or 600-fold, or 700 fold, or 1000-fold, relative to an unmodified humanized antibody. Because of increased ADCC activity, such modified antibodies can be used at lower dosages than their unmodified counterparts and generally have fewer or reduced side effects in patients.

### Complement Dependent Cytotoxicity

An antibody as described herein can be used in complement-dependent cytotoxicity (CDC). CDC involves the central innate complement system which acts as the effector of adaptive immunity. The classical CDC pathway is triggered by antibody molecules binding to an antigen on a target cell and is initiated by binding of a C1q protein to the Fc domain of the bound antibody. The resulting complement cascade activates a membrane attack pathway, leading to the formation of a membrane attack complex which induces lysis of the target cell. An antibody as described herein can be modified to enhance its capability to trigger CDC by any method known in the art, such as but not limited to, engineering the protein backbone to contain amino acid residue substitutions in the constant domains of the antibody heavy chain. For an example of a combination of IgG1 amino acid substitutions used to enhance CDC activity, see Moore et al., mAbs, 2(2), 181-189 (2010). The CDC activity of a modified antibody as described herein can be enhanced by, for example, 10-fold, or 20-fold, or 30-fold, or 40-fold, or 50-fold, or 100-fold, or 500-fold, or 600-fold, or 700 fold, or 1000-fold, relative to an unmodified humanized antibody.

### Antigens

An “antigen” is an entity (e.g., a proteinaceous entity or peptide) to which an immunoglobulin or antibody (or antigen-binding fragment thereof) specifically binds.

An antibody of the present invention can bind to the antigenic 15-amino acid SP70 peptide epitope (SEQ ID NO:12) of EV71 (Example 4). The SP70 epitope is located on the VP1 capsid protein of EV71 at residues 208-222. The amino acid sequence of SP70 is highly conserved amongst the VP1 sequences of EV71 strains from various sub-genotypes of the virus. Thus EV71, a non-enveloped, single stranded, positive-sense RNA virus comprising the SP70 epitope (SEQ ID NO:12), can be neutralized by an antibody of the present invention binding to the SP70 epitope.

### Enterovirus 71

EV71 and Coxsackievirus A16 (CA16) are non-enveloped, single stranded, positive-sense RNA viruses, belonging to the enterovirus genus of the picornavirus family (which also includes viruses such as poliovirus). The human enterovirus species contains four serotypes (A-D) of which EV71 and CA16 belong to serotype A.

EV71 has several genotypic groups; A, B, C, D, E and F. Genotypes B and C can be further broken down into sub-genotypes, B1-B5 and C1-C5, with each sub-group exhibiting ˜15% divergence from the others. The different strains of EV71 belonging to these sub-genotypes are documented for large outbreaks of HFM D. Group A consists of only one member which was first identified in California, USA in 1970 and is considered to no longer be circulating. The B genotype has been prominent in Malaysia and Singapore, whereas the C genotype has been shown to be prominent in China and Vietnam. Bessaud et al (PLOS ONE; March 2014, Volume 9, Issue 3, e90624) report three additional genotypic groups, including one Indian genotypic group (genogroup D) and 2 African ones (E and F).

EV71 and CA16 both have a relatively high rate of genomic evolution and new sub-genotypes keep arising. A divergence of 17-22% is the cut-off for designation of a new genotype and EV71 has diverged into two genotypes within the last 40 years. In China only the C4 sub-genotype of EV71 is endemic and although the molecular basis is unclear, the C4a evolutionary branch appears to show higher morbidity and mortality and greater neurovirulence (Zhang et al., PLoS One, 2011; Zhang et al., J. Clin Microbiol, 2010).

The structure of all human enteroviruses is similar and therefore EV71 and CA16 both have a similar structure, composed of small icosahedral particles which contain a single-stranded, positive-sense, polyadenylated virus RNA of approximately 7.4 kb. Each protomer in the virus capsid contains a copy of the four structural viral proteins (VP1-VP4), of which VP1, VP2 and VP3 are external whereas VP4 is completely internalized and is therefore not exposed to the host antibody response. All of these structural proteins are encoded by the P1 region of the genome. VP1 is the major capsid protein of EV71 and is clustered with neutralization epitopes, including the SP70 epitope where the antibodies of the present invention bind and neutralize the virus. Thus, antibodies of the present invention are effective against EV71, where EV71 is a picornavirus having a single-stranded, positive-sense, polyadenylated genome of approximately 7.4 kb, with a major capsid protein (VP1) that comprises the SP70 epitope that is SEQ ID NO:12.

In the entry process, the virus first attaches to permissive cells by binding to a cellular receptor. It is well recognized that EV71 can infect neurons in humans and in animal models, and causes neuroinflammation in the CNS which leads to neuroencephalitis, aseptic meningitis, and brain stem encephalitis (reviewed by Weng KF et al., Microbes Infect, 2010). Following binding a series of structural changes occur in the virus capsid and pores are formed in the cell membrane allowing the virus to cross the plasma membrane via endocytosis, and then undergo a capsid conformational change to release viral RNA.

The frequency of neurological complications has been shown to vary between Asian outbreaks and it is not known what factors determine whether EV71 or CA16 infection will cause neurological complications. However, it has been suggested that specific strains of the virus could be responsible for the pathogenesis of severe neurological disease. In 1999 subgroups B3 and C2 were reported to be circulating in Australia. In 1998, isolates from children in Taiwan with severe neurological disease were shown to be from the C2 subgroup. In contrast, isolates from children with uncomplicated HFMD in Sarawak in the epidemic of 1997 were shown to be from the B3 subgroup. This suggested that EV71 viruses from the C2 subgroup may be more likely to cause CNS complications, although this is still to be fully confirmed. However, more recently in China it is the C4a subgroup that is reported to be more neurovirulent.

The high incidence of HFMD in Asia compared to the rest of the world has led to investigations into the possibility of host susceptibility for HFMD. A genetic study carried out by Chang and colleagues reported that HLA-A33 was associated with increased susceptibility to EV71 infection and that HLA-A33 was more frequent in Asian populations than in white populations (Chang LY et al., Pediatrics, 2008). The study also suggested that HLA-A2 may be linked to the risk of cardiopulmonary failure in EV71 infected patients, although the mechanism for this is unknown. Another theory put forward for the neurological complications associated with HFMD is dual infection, which has been shown for HFMD. Dual infection of the EV71 B3 subgroup and adenovirus type 21 was reported in the outbreak in Sarawak in 1997 (Ooi MH et al., Clin Infect Dis 2007). Dual infection of EV71 with Dengue fever and Japanese encephalitis has also been reported (Ooi et al., 2007). However at present there is no conclusive evidence to suggest that dual infection is responsible for CNS involvement in HFMD.

Antibodies against the EV71 capsid protein have been shown to bind to the virus surface and therefore occupy receptor binding sites and block virus/receptor interaction. This results in the inhibition of virus entry into the cell (termed neutralization). A mouse monoclonal antibody, D5, exhibits potent neutralization effects on EV71 and supports the above mechanism of action. D5 has an 1095 of 0.3 pg/ml (Ku Z et al., J Virol Methods, 2012) and can inhibit EV71 binding to permissive cells. In addition, D5 can neutralize EV71 at a post-attachment step.

So far, no vaccines or specific (chemical or biochemical) antiviral drugs have been approved for HFMD treatment and therefore symptomatic approaches to relieve fever and sores and supportive care with adequate fluid intake represent the only available course of treatment for the disease at present. In Western countries, doctors allow the disease to run its course. However, severe complications are more common in Asian outbreaks, as discussed above.

From its virus neutralization capability, the mouse D5 antibody was considered by the present inventors to be a candidate for humanization to produce antibodies suitable for therapeutic uses in humans.

The antibodies of the present invention offer therapeutic benefits in addition to preventative benefits, and are thus more useful in treating HFMD than the vaccination approaches which have been pursued to date. A vaccine to protect against HFMD would also only be effective in preventing outbreaks of HFMD if enough of the public choose to have the optional vaccination. An antibody of the present invention would provide prophylactic treatment, but would also provide beneficial therapy once individuals contract the disease.

### Compositions

### Pharmaceutical Compositions

An antibody of the invention can be formulated and/or administered as a pharmaceutical composition comprising the active therapeutic antibody agent and a variety of other pharmaceutically acceptable components, see Remington: The Science and Practice of Pharmacy (22nd ed., Pharmaceutical Press, London, Pa. (2013)). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions containing an antibody of the invention can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, an antibody or composition of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation, which can be formulated in such a manner as to permit a sustained release of the active ingredient.

The term parenteral as used herein includes subcutaneous, intravenous, intradermal, intramuscular, intraperitoneal, and intrathecal administration of an antibody or composition of the invention. An antibody or composition of the invention may also be administered by nasal or gastric methods.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249: 1527 (1990) and Hanes, Advanced Drug Delivery Reviews 28:97 (1997)). The agents of this invention can be administered in the form of a depot injection or implant preparation, which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein.

Alternatively, transdermal delivery can be achieved using a skin patch or using transferosomes (Paul et al., Eur. J. Immunol. 25:3521 (1995); Cevc et al., Biochem. Biophys. Acta 1368:201-15 (1998)).

Compositions of the present invention can comprise an antibody of the present invention, pharmaceutically acceptable carriers as described herein, and other therapeutic agents, in particular prophylactic or therapeutic agents useful for the prevention, management or treatment of HFMD, an HFMD-related disease, and/or infection with the EV71 virus. Such therapeutic agents can comprise analgesic drugs, anti-inflammatory drugs, anti-viral drugs, drugs which ameliorate fever or elevated body temperature, and therapeutic compounds designed to numb pain, e.g., mouthwashes or sprays which can numb mouth pain. A composition of the present invention can additionally comprise compositions for rehydrating a subject, for example by intravenous therapy. A composition of the present invention can also comprise an antibody of the present invention and at least one other antibody which is not of the present invention, such as an antibody that binds another infectious agent, e.g., an antibody that can bind to the Coxsackievirus A16 (CA16) virus.

Compositions of the present invention can comprise nucleic acids, i.e., DNA or RNA, encoding the antibodies disclosed herein, and any method of delivery of such nucleic acids, with or without any of the other composition compounds discussed above. Compositions can also comprise vectors, for example but not limited to, the expression vectors described herein, themselves comprising the nucleic acids of the present invention.

Compositions of the present invention can comprise viral vectors, for use as nucleic acid delivery systems into cells. Suitable viral vector nucleic acid delivery systems include retroviral systems, adenoviral vectors, viral vectors from the pox family including vaccinia virus and the avian pox viruses, and viral vectors from the alpha virus genus. A nucleic acid encoding an antibody of the present invention, or a vector containing the same, can be packaged into liposomes for delivery to an individual or cell, which can be incorporated into compositions as described. Vectors and nucleic acids encoding an antibody can also be adsorbed to or associated with particulate carriers.

Compositions of the present invention can comprise gene therapy vectors which contain nucleotide sequences encoding for the antibodies of the present invention, or naked antibody polypeptide chains according to the invention. Compositions can comprise such vectors or polypeptides in combination with the antibodies of the present invention, and any other composition components described above.

### Kits

An antibody of the present invention may be used in a kit. The term “kit” is used in reference to a combination of reagents and other materials which facilitate sample analysis. In some embodiments, an immunoassay kit of the present invention includes a suitable antigen, binding agent comprising a detectable moiety, and detection reagents. A system for amplifying the signal produced by detectable moieties may or may not also be included in the kit. Furthermore, in other embodiments, the kit includes, but is not limited to, components such as apparatus for sample collection, sample tubes, holders, trays, racks, dishes, plates, instructions to the kit user, solutions or other chemical reagents, and samples to be used for standardization, normalization, and/or control samples.

Kits may contain at least one antibody of the present invention. In one embodiment, a kit comprises a composition of the present invention, in one or more containers. In another embodiment, a kit comprises a composition according to the present invention, in one or more containers, and one or more other prophylactic or therapeutic agents useful for the prevention, management or treatment of HFMD, an HFMD-related disease, and/or infection with the EV71 virus. If the composition containing components for administration is not formulated for delivery via the alimentary canal, such as oral delivery, a device capable of delivering the kit components through some other route may be included, e.g., a syringe. The kit may further include instructions for preventing, treating, managing or ameliorating HFMD, an HFMD-related disease, and/or infection with the EV71 virus, as well as side effects and dosage information for method of administration.

The present invention also provides diagnostic kits. Antibodies of the present invention can be useful for monitoring, diagnosing, or providing a prognosis for the development or progression of HFMD, an HFMD-related disease, and/or infection with the EV71 virus, and can be used in a kit suitable for such purposes. An antibody of the present invention can be used in a diagnostic kit to detect the presence of the EV71 antigen, or the SP70 peptide epitope of the EV71 antigen, in a sample of body fluid taken from an individual, where the individual may be a human, or a mammal, such as a non-human primate or a laboratory animal, including mice, rats and rabbits. A sample of body fluid, such as but not limited to blood, serum or cerebrospinal fluid, is taken from an individual and tested for the presence of antigen using the antibodies of the present invention. Measuring EV71 levels in the blood of an individual using an antibody of the invention can provide information about suitable administration schedules and dose of an antibody or composition of the invention for treating such an individual. The methods above are generally performed in vitro.

A kit which is useful for the diagnosis described above can comprise antibodies of the present invention which are coupled to a detectable substance including, but not limited to: various enzymes for use in assays including EIA and ELISA, such as, but not limited to, horseradish peroxidase, alkaline phosphatase, beta-galactosidase, or acetylcholinesterase; prosthetic groups, such as, but not limited to, streptavidin/biotin and avidin/biotin; particles, such as latex beads or bacteria, for use in agglutination tests; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as, but not limited to, luciferase, luciferin, and aequorin; radioactive materials, such as, but not limited to, iodine (¹³¹I, ¹²⁵I, ¹²³I, 121I), carbon (¹⁴C), sulfur (³⁵S), tritium (³H), indium (¹¹⁵In, ¹¹³In, ¹¹²In), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Sn; positron-emitting metals using various positron-emission tomographies, non-radioactive paramagnetic metal ions, and molecules that are radiolabelled or conjugated to specific radioisotopes. Any detectable label that can be readily measured can be conjugated to an antibody of the present invention and used in diagnosing a disease as described herein. The detectable substance may be coupled or conjugated either directly to an antibody or indirectly, through an intermediate (such as, for example, a linker known in the art) using techniques known in the art. See, e.g., U.S. Pat. No. 4,741,900 for metal ions which can be conjugated to antibodies for use as a diagnostics.

Detection of the antigen by using any of the methods or detectable substances described above can give a positive result for the presence of the EV71 virus or the SP70 peptide using the antibodies of the present invention in a kit as described herein and can diagnose an individual as having HFMD, an HFMD-related disease, and/or infection with the EV71 virus. Such an individual may subsequently require and/or undergo treatment for HFMD as described herein.

### Therapy

The present invention is directed inter alia to the treatment of Enterovirus 71 (EV71) and related diseases and disorders, including hand, foot, and mouth disease (HFMD). The present invention is also directed to a method of treatment or prophylaxis of HFMD by administering to an individual in need of treatment an effective amount of an antibody or composition of the present invention described herein. An antibody or composition, preferably a pharmaceutical composition (e.g., a composition comprising an antibody of the invention and one additional therapeutic agent, such as an antibody that can bind to the Coxsackievirus A16 (CA16) virus), of the present invention can be for use in a method of treatment of the human or animal body. An antibody or composition, preferably a pharmaceutical composition, of the present invention can be for use in a method of treatment of the human or animal body, wherein the treatment is therapeutic or prophylactic treatment of HFMD in an individual.

The treatment methods mentioned above can comprise administration of the antibody or composition (e.g., a composition comprising an antibody of the invention and one additional therapeutic agent, such as an antibody that can bind to the Coxsackievirus A16 (CA16) virus) of the present invention to an individual under conditions that generate a beneficial therapeutic response in the individual e.g., for the prevention or treatment of HFMD.

Such an individual can be infected with EV71 or may be suffering from HFMD or an HFMD-related disease. The methods of treatment described herein can be used on both asymptomatic patients, and those currently showing symptoms of disease, particularly HFMD. An antibody of the invention may be administered prophylactically to an individual who is not infected with EV71 and does not have HFMD. An antibody of the invention may be administered to an individual who is infected with EV71 and does not have, or does not exhibit the symptoms of, HFMD. An antibody of the invention may be administered to an individual who does have, or appears to have, HFMD or an HFMD-related disease. Such an individual may be one in which is it not known whether or not EV71 is present. Individuals amenable to treatment include individuals at risk of contracting an EV71- or HFMD-related disorder but not showing symptoms and individuals suspected of having an EV71- or HFMD-related disorder, as well as individuals presently showing symptoms. Antibodies of the present invention can be administered prophylactically to the general population without the need for any assessment of the risk of the subject individual.

The terms “treat”, “treating” or “treatment” (or grammatically equivalent terms) mean that the severity of the individual's condition is reduced or at least partially improved or ameliorated and/or that some alleviation, mitigation or decrease in at least one clinical symptom is achieved and/or there is an inhibition or delay in the progression of the condition and/or prevention or delay at the onset of a disease or illness.

An antibody of the invention which can be used in a method of treatment for EV71 infection and/or HFMD or an HFMD-related disease can be an antibody of any sequence and format described herein that specifically binds to the SP70 peptide epitope of EV71. The antibodies used for methods of treatment as described herein can be fragments of antibodies of the present invention, for example antigen binding fragments. An antibody of the invention can be administered to an individual infected with EV71 from any genotypic group, A, B, C, D, E or F which includes EV71 classified into any sub-genotypic group, such as B1-B5 or C1-C5.

An antibody according to the present invention can be administered to an individual in need of treatment with a pharmaceutical carrier or pharmaceutical composition, or in any composition described herein.

Alternatively, the antibody can be administered to an individual by administering a polynucleotide encoding at least one antibody chain. The polynucleotide is expressed to produce the antibody chain in the patient. Optionally, the polynucleotide encodes heavy and light chains of the antibody. The polynucleotide is expressed to produce the heavy and light chains in the individual.

An antibody of the present invention can be used in a method of preventing or treating HFMD or related diseases or disorders that involves administering to the patient an effective dosage of the antibody as described herein. As used herein, an “effective amount” or an “effective dosage” or a “sufficient amount” (or grammatically equivalent terms) of a therapeutic antibody of the invention refers to an amount of antibody or composition of the invention that is effective to produce a desired effect, which is optionally a therapeutic effect (i.e., by administration of a therapeutically effective amount). For example, an “effective amount” or an “effective dosage” or a “sufficient amount” can be an amount so that the severity of the individual's condition, e.g., HFMD, is reduced or at least partially improved or ameliorated and/or that some alleviation, mitigation or decrease in at least one clinical symptom is achieved and/or there is an inhibition or delay in the progression of the condition and/or prevention or delay at the onset of a disease or illness, e.g., HFMD.

The terms “patient”, “individual” or “subject” include human and other mammalian subjects that receive either prophylactic or therapeutic treatment with one or more agents (e.g., immunotherapeutic agents or antibodies) of the invention. Mammalian subjects include primates, e.g., non-human primates. Mammalian subjects also include laboratory animals commonly used in research, such as but not limited to, rabbits and rodents such as rats and mice. Mammalian subjects can also be domestic animals, including bovine, ovine, equine, and porcine livestock, and pets such as cats and dogs.

### Dosage

An amount of an antibody or composition of the present invention adequate to accomplish therapeutic or prophylactic treatment is defined as an effective dose, e.g., a therapeutically- or prophylactically-effective dose. In both prophylactic and therapeutic treatment regimes, reagents may be administered in several dosages until a sufficient immune response has been achieved. The term “immune response” or “immunological response” includes the development of a humoral (antibody mediated) and/or a cellular (mediated by antigen-specific T cells or their secretion products) response directed against an antigen in a recipient subject. Typically, the immune response is monitored and repeated dosages are given if the immune response starts to wane.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but non-human mammals, e.g., non-human primates, rabbits, rats and mice, including transgenic mammals, can also be treated. Treatment dosages need to be titrated to optimize safety and efficacy.

For passive immunization with an antibody of the present invention, the dosage ranges from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. In another example, dosages can be 0.5 mg/kg body weight or 15 mg/kg body weight or within the range of 0.5-15 mg/kg, preferably at least 1 mg/kg. In another example, dosages can be 0.5 mg/kg body weight or 20 mg/kg body weight or within the range of 0.5-20 mg/kg, preferably at least 1 mg/kg. In another example, dosages can be 0.5 mg/kg body weight or 30 mg/kg body weight or within the range of 0.5-30 mg/kg, preferably at least 1 mg/kg. In a preferred example, dosages can be about 30 kg/mg.

The methods of the invention may comprise the administration of an antibody to a subject as a single dose, in two doses, or in multiple doses. The dose of the antibody may be from about 100 μg/kg to 100 mg/kg body weight of the patient, from about 300 μg/kg to 30 mg/kg body weight of the patient, or from about 1 mg/kg to 10 mg/kg body weight of the patient. Subjects can be administered such doses daily, on alternative days, weekly or according to any other schedule determined by empirical analysis. A treatment may involve administration in multiple dosages over a prolonged period, for example, of at least six months. Additional treatment regimes may involve administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies, at least one antibody according to the present invention, with different binding specificities may be administered simultaneously, in which case the dosage of each antibody administered falls within the ranges indicated.

An antibody of the present invention may be administered on multiple occasions. Intervals between single dosages can be weekly, monthly or yearly. Intervals can also be irregular as indicated by measuring blood levels of antibody to EV71 in the patient. In some methods, dosage is adjusted to achieve a plasma antibody concentration of 1-1000 μg/ml and in some methods 25-300 μg/ml. Alternatively, an antibody of the present invention can be administered as a sustained release formulation, in which case less frequent administration is required. Dosage and frequency vary depending on the half-life of the antibody in the patient. In general, humanized antibodies show a longer half-life than chimeric and nonhuman antibodies.

The dosage and frequency of administration can vary depending on whether the treatment is prophylactic or therapeutic. In prophylactic applications, compositions containing the antibodies of the present invention or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time.

In therapeutic applications, a relatively high dosage (e.g., from about 1 to 200 mg of antibody per dose, with dosages of from 5 to 25 mg being more commonly used) at relatively short intervals is sometimes required until progression of the disease is reduced or terminated, and preferably until the patient shows partial or complete amelioration of symptoms of disease.

Doses for nucleic acids encoding antibodies of the present invention range from about 10 ng to 1 g, 100 ng to 100 mg, 1 μg to 10 mg, or 30-300 μg DNA per patient. Doses for infectious viral vectors vary from 10-100, or more, virions per dose.

Antibodies and compositions of the invention can be administered for therapeutic and/or prophylactic treatment by parenteral, topical, intravenous, oral, gastric, subcutaneous, intra-arterial, intracranial, intraperitoneal, intranasal or intramuscular methods, as described herein. Intramuscular injection or intravenous infusion are preferred for administration of antibodies.

### Administration of Nucleic Acids

Immune responses against EV71 can be induced by administration of nucleic acids encoding antibodies of the present invention. Such nucleic acids can be DNA or RNA as described within, and with reference to the sequence listing. A nucleic acid segment encoding an antibody of the present invention can be linked to regulatory elements, such as a promoter and enhancer, that allow expression of the nucleic acid segment in the intended target cells of a patient. For expression in blood cells, as is desirable for induction of an immune response, exemplary promoter and enhancer elements include those from light or heavy chain immunoglobulin genes and/or the CMV major intermediate early promoter and enhancer (Stinski, U.S. Pat. Nos. 5,168,062 and 5,385,839). The linked regulatory elements and coding sequences are often cloned into a vector. For administration of double-chain antibodies, the two chains can be cloned in the same or separate vectors.

A number of viral vector nucleic acid delivery systems are available including retroviral systems (see, e.g., Lawrie and Tumin, Cur. Opin. Genet. Develop. 3:102-109 (1993)); adenoviral vectors (see, e.g., Bett et al., J. Virol. 67:5911 (1993)); adeno-associated virus vectors (see, e.g., Zhou et al., J. Exp. Med. 179:1867 (1994)), viral vectors from the pox family including vaccinia virus and the avian pox viruses, viral vectors from the alpha virus genus such as those derived from Sindbis and Semliki Forest Viruses (see, e.g., Dubensky et al., J. Virol. 70:508 (1996)), Venezuelan equine encephalitis virus (see Johnston et al., U.S. Pat. No. 5,643,576) and rhabdoviruses, such as vesicular stomatitis virus (see Rose, U.S. Pat. No. 6,168,943) and papillomaviruses (Ohe et al., Human Gene Therapy 6:325 (1995); Woo et al., WO 94/12629 and Xiao & Brandsma, Nucleic Acids. Res. 24, 2630-2622 (1996)).

A nucleic acid encoding an antibody of the invention, or a vector containing the same, can be packaged into liposomes for delivery to an individual or cell. Suitable lipids and related analogs are described by Eppstein et al., U.S. Pat. No. 5,208,036, Feigner et al., U.S. Pat. No. 5,264,618, Rose, U.S. Pat. No. 5,279,833, and Epand et al., U.S. Pat. No. 5,283,185. Vectors and nucleic acids encoding such antibodies can also be adsorbed to or associated with particulate carriers, examples of which include polymethyl methacrylate polymers and polylactides and poly (lactide-co-glycolides), see, e.g., McGee et al., J. Micro Encap. (1996).

Gene therapy vectors which contain nucleic acids of the present invention or naked polypeptides can be delivered in vivo by administration to an individual patient, typically by systemic administration (e.g., intravenous, intraperitoneal, nasal, gastric, intradermal, intramuscular, subdermal, or intracranial infusion) or topical application (see e.g., Anderson et al., U.S. Pat. No. 5,399,346). The term “naked polynucleotide” refers to a polynucleotide not delivered in association with a transfection facilitating agent. Naked polynucleotides are sometimes cloned in a plasmid vector. Such vectors can further include facilitating agents such as bupivacaine (Weiner et al., U.S. Pat. No. 5,593,972). DNA can also be administered using a gene gun. See Xiao & Brandsma, supra. The DNA encoding the antibody is precipitated onto the surface of microscopic metal beads. The microprojectiles are accelerated with a shock wave or expanding helium gas, and penetrate tissues to a depth of several cell layers. For example, The Accel™ Gene Delivery Device manufactured by Agricetus, Inc. Middleton Wis. is suitable. Alternatively, naked DNA can pass through skin into the blood stream simply by spotting the DNA onto skin with chemical or mechanical irritation (see Howell et al., WO 95/05853).

In a further variation, vectors encoding antibodies of the present invention can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

### Nucleic Acids, Host Cells and Production of Antibodies

The present invention is further directed to nucleic acids which encode antibody polypeptide chains as disclosed. Nucleic acids of the present invention can have nucleotide sequences according to SEQ ID NO:21, SEQ ID NO:22 and SEQ ID NO: 23. The nucleic acids may also be RNA sequences, wherein the thymine nucleobases are substituted with uracil.

Antibodies of the present invention may be produced by recombinant expression. Nucleic acids as described above, encoding light and heavy chain variable regions optionally linked to constant regions, can be inserted into expression vectors. Vectors which comprise nucleic acids encoding antibodies of the invention are themselves a part of the present invention. The light and heavy chains can be cloned in the same or different expression vectors. The nucleic acids encoding antibody chains of the invention are operably linked to one or more control sequences in the expression vector(s) that ensure the expression of antibody polypeptides. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells (e.g., COS, CHO, or Expi293 cells). Such vectors can be incorporated into an appropriate host, whereby the host is maintained under conditions suitable for high level expression of the nucleotide sequences, and the collection and purification of the antibodies.

In one embodiment, the nucleic acid encodes an antibody of the invention, as described herein. In another embodiment, a vector, preferably an expression vector, comprises one or more nucleic acids that encode an antibody of the invention. In a further embodiment, a vector comprises one or more nucleic acids that encode an antibody of the invention, operably linked to a promoter. Exemplary expression vectors are pHuK and pHuG1 which, in combination with the nucleic acids disclosed herein, comprise nucleotide sequences encoding the antibodies of the present invention. Other vectors which provide nucleotide sequences encoding the constant regions of antibody light and heavy chains can also be used.

The expression vectors for use in the present invention are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences (see, e.g., Itakura et al., U.S. Pat. No. 4,704,362).

Host cells are transformed with the expression vectors and cultured in conventional nutrient media as appropriate for inducing promoters, selecting transformants, or amplifying the genes encoding the required sequences. A host cell comprising a nucleic acid or vector of the present invention is itself a part of said invention.

The present invention is also directed towards a method for making an antibody according to the invention, the method comprising expressing, in a host cell culture, a vector also according to the invention to produce said antibody, and recovering the antibody from the cell culture. This method involves transferring a vector comprising one or more nucleic acids encoding an antibody or antibody chains, as described above, into a host cell, as described herein, growing the host cell culture under conditions which allow for expression of the nucleic acid(s) and recovering the expressed antibody by any suitable method known in the art. In one example, the vectors are pHuK and pHuG1, the nucleic acids comprise the nucleotide sequences set out in SEQ ID NO:22 and SEQ ID NO:23, and the host cells are human Expi293 suspension cells (see Example 1).

Microbial host organisms suitable for use in cloning the nucleic acids of the present invention include prokaryotic hosts; Escherichia coli, bacilli, such as Bacillus subtilis, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation. Vectors for use in prokaryotic cells also require an origin of replication component.

Other microbes, such as yeast, may also be used to express the antibodies, nucleic acids or vectors of the present invention. Saccharomyces is a preferred yeast host, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian tissue cell culture may also be used to express and produce the antibody polypeptides of the present invention (e.g., polynucleotides encoding antibodies or fragments thereof) from nucleic acids and vectors of this invention. See Wnnacker, From Genes to Clones, VCH Publishers, N.Y., N.Y. (1987). A eukaryotic or mammalian cell host comprising a nucleic acid or vector of the invention is itself part of the present invention. Eukaryotic cells are actually preferred, because a number of suitable host cell lines capable of secreting heterologous proteins (e.g., intact antibodies) have been developed in the art, and include CHO cell lines, various Cos cell lines, HeLa cells, myeloma cell lines, or transformed B-cells or hybridomas. The cells can be human or non-human e.g. non-human mammalian cells. Preferably, the cells are Expi293 human cells. The antibodies of the present invention can be produced in cell lines engineered to produce afucosylated proteins, such as the Potelligent® CHOK1SV cell line (BioWa/Lonza), GlymaxX®-engineered cells (ProBioGen) or the duck embryonic stem cell line EB66 (Valneva). Expression vectors for mammalian cells generally include, but are not limited to, one or more of the following: a signal sequence, one or more marker genes, an enhancer element, a promoter, and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

A vector of the present invention for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

Alternatively, antibody-coding sequences of the present invention can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

Vectors of the present invention containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Green and Sambrook, Molecular Cloning: A Laboratory Manual (Cold Spring Harbor Press, 4th ed., 2012). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

When heavy and light chains are cloned on separate expression vectors, the vectors are co-transfected to obtain expression and assembly of intact antibodies of the present invention. Once expressed, the whole antibodies, their dimers, individual light and heavy chains, or other immunoglobulin forms of the present invention can be purified according to standard procedures of the art, including ammonium sulphate precipitation, affinity columns, column chromatography, HPLC purification, gel electrophoresis and the like (see generally Scopes, Protein Purification (Springer-Verlag, N.Y., (1982)). Substantially pure antibodies of at least about 90 to 95% homogeneity are preferred, and 98 to 99% or more homogeneity are most preferred, for pharmaceutical uses as described herein. Standard protein purification methods known in the art can be employed. The following procedures are exemplary of suitable protein purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulphate precipitation, and gel filtration.

Production of the antibodies of the invention can be carried out by any suitable technique including techniques described herein as well as techniques known to those skilled in the art. Antibodies of the invention can be produced on a commercial scale using methods that are well-known in the art for large scale manufacturing of antibodies. For example, this can be accomplished using recombinant expressing systems such as those described herein.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the description and examples should not be construed as limiting.

The present invention is illustrated by the following examples.

### EXAMPLES

The examples herein describe the generation and properties of a humanized version of the anti-EV71 D5 antibody from mouse.

### Example 1. Design of D5 Humanized Antibody Variants

Preliminary investigations illustrated that the binding capacity of a chimeric D5 antibody was similar to that of the natural murine D5 antibody. As the chimeric D5 antibody was found to retain function and binding to the EV71 virus, further humanization of the antibody was undertaken.

cDNA encoding the variable regions from murine D5 antibody Kappa light chain (VH) and heavy chain (VK) was amplified and introduced into kappa/IgG expression vectors (pHuK and pHuG1 respectively). The constructs were co-transfected into Expi293 suspension cells to generate chimeric D5 (cD5) antibodies, comprising human constant regions and murine D5 variable regions.

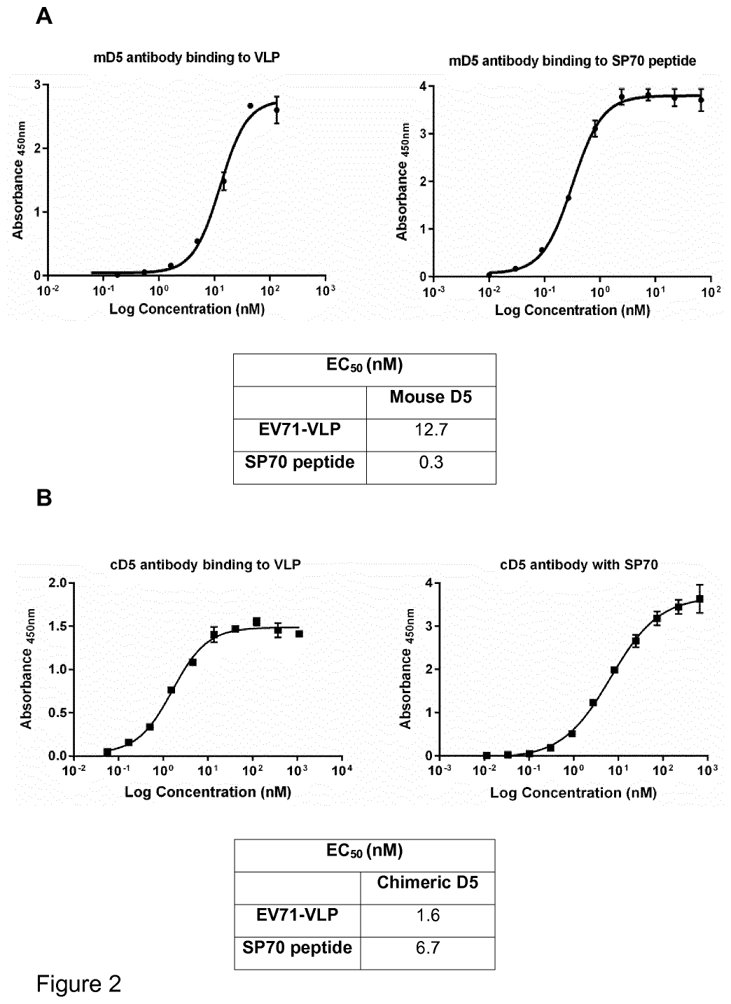

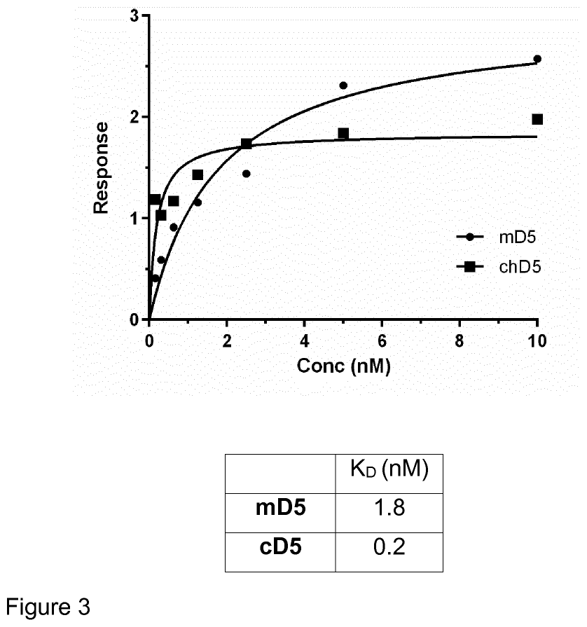

Binding of the chimeric D5 antibody to the EV71-VLP (virus-like particle) and the SP70 peptide binding epitope was measured by ELISA, and the chimeric antibody was found to have comparable, if slightly lower, EC₅₀ values than the murine D5 antibody (FIGS. 2a and 2b). Further characterisation of the binding of mouse and chimeric D5 antibodies to EV71-VLP was performed by Bio-Layer Interferometry. The steady state plot in FIG. 3, of mouse and chimeric D5 antibodies binding to EV71-VLP, shows that the two antibodies bind EV71-VLP with comparable apparent K_{D} values.

Humanization requires the identification of suitable human variable regions as framework regions for non-human complementarity determining regions (CDRs). Human variable heavy (VH) and light (VK) chain sequence databases were interrogated with D5 antibody VH and VK protein sequences. Framework residues within 4Å of the CDR residues in the structures of the mouse D5 antibody were identified, and designated as the “4Å Proximity Residues”. Human VH sequence alignments with highest identity to D5 VH in the 4Å Proximity Residues were considered as suitable framework sequences.

The sequence EF178053 was chosen as the human heavy chain donor candidate due to high sequence identity with murine D5 VH. CDRs 1, 2 and 3 from the murine D5 antibody heavy chain were introduced into the framework of EF178053 to create a sequence designated D5 HA (SEQ ID NO:1). Eight identified 4A Proximity Residues were back-mutated to the equivalent mouse residue to create a humanized antibody variant designated D5 HB (SEQ ID NO:11) containing eight mutations: Y27F; T28N; F291; T3OK; R67K; V68A; R72A and S98N.

Eight further heavy chain variants were created, designated HC (SEQ ID NO:13), HD (SEQ ID NO:14), HE (SEQ ID NO:15), HF (SEQ ID NO:16), HG (SEQ ID NO:17), HH (SEQ ID NO:18), HI (SEQ ID NO:19) and HJ (SEQ ID NO:20), each comprising one of the eight mutations in the order above (i.e. variant HC contains the Y27F mutation only, HD contains the T28N mutation only, and so forth), see FIG. 1.

The sequence AJ388646 was chosen as the human kappa light chain donor candidate. CDRs 1, 2 and 3 from murine D5 VK were introduced into the framework of AJ388646. The resulting construct was designated D5 KA (SEQ ID NO:2). Another variant, D5 KB (SEQ ID NO:7), was created in which two unmatched 4Å Proximity Residues in D5 KA were back-mutated (I2V and V3L) to the equivalent mouse residue. Three further variants were created with substituted KA domains, designated KC (SEQ ID NO:8; 12V), KD (SEQ ID NO:9; V3L) and KE (SEQ ID NO:10; 12V, V3L and Q50K). Sequences and mutations are shown in FIG. 4.

Humanized antibodies discussed in the following examples are described as “hEV71 [heavy chain variant][light chain variant]”. For example, “hEV71 HBKA” is the humanized form of the D5 antibody (binding the EV71 virus), containing the HB heavy chain and the KA light chain.

### Example 2. Generation of D5 Humanized Antibodies

The modified variable domain nucleotide sequences of EF178053 (HA to HJ) and AJ388646 (KA to KE) created in Example 1 were introduced into expression vectors pHuK and pHuG1 and expressed in E. coli cells. Plasmid DNA was extracted and expression plasmid preparations encoding humanized VH and VK chains were used to transfect Expi293 cells. Cells were cultured for 5-7 days in serum-free media and the resulting secreted antibody was harvested.

### Example 3. Efficacy of Initial D5 Humanized Antibody Variants

This example shows that the humanized D5 antibody variants created in Example 1 showed lower binding with the EV71-VLP and substantially lower binding with the associated EV71 SP70 peptide D5 epitope, when compared to the chimeric D5 antibody.

Binding of the HA/HB heavy chains in combination with KA/KB light chain versions of the humanized D5 antibody to EV71-VLP or SP70 peptide antigens was performed by binding ELISA (as described in Ku et al., (2012) J. Virol Methods 71 186:193-197). Serial dilutions of antibody variants (starting from ˜100 −g/ml) were added to immobilised EV71-VLP or the SP70 peptide epitope, shaken for one hour at room temperature and washed with PBS-T. Anti-human kappa chain HRP was added and the incubation and washing steps were repeated. TMB/K-Blue substrate was incubated with the reaction for 5-10 minutes at room temperature before the reaction was stopped with H₂SO₄ RED STOP. The optical density was read at 450/650 nm.

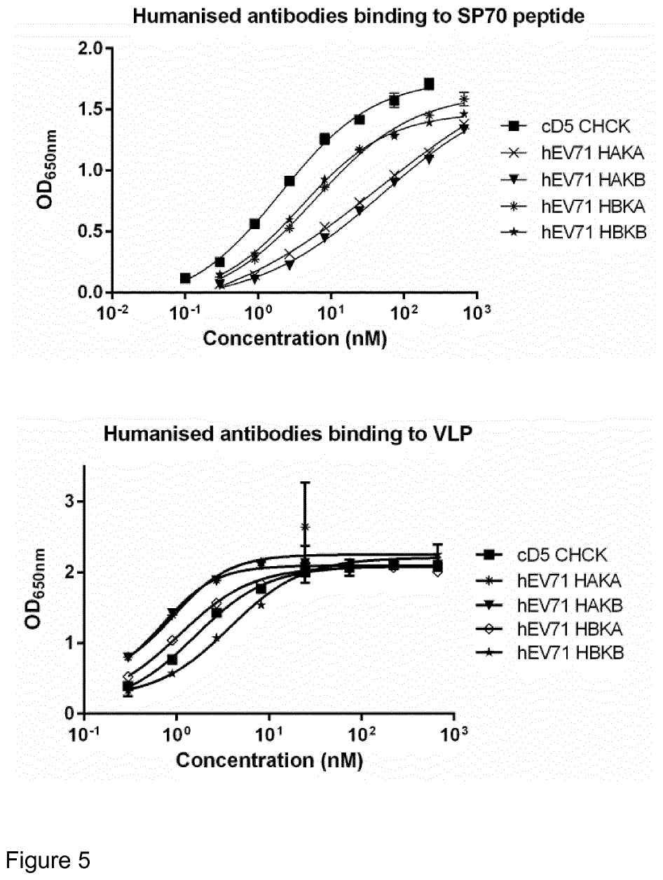

FIG. 5 shows that the presence of KA or KB versions of the kappa light chain made no observable difference in the binding capacity of the antibody to the antigens. The two back-mutations introduced into KB appear not to be essential for antibody binding.

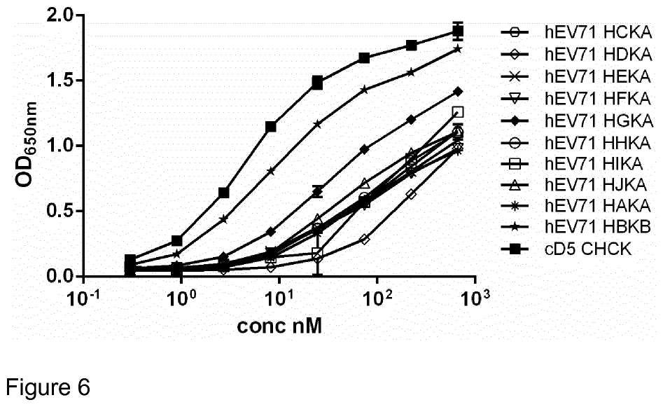

Humanized antibody variants containing the HA and HB heavy chains showed decreased binding capability to the EV71 SP70 peptide epitope when compared to the chimeric D5 antibody (FIG. 5). FIG. 6 shows that the binding capability of further humanized D5 antibody versions hEV71 HCKA to hEV71 HJKA to the SP70 peptide epitope was severely impaired when compared to the chimeric D5 antibody.

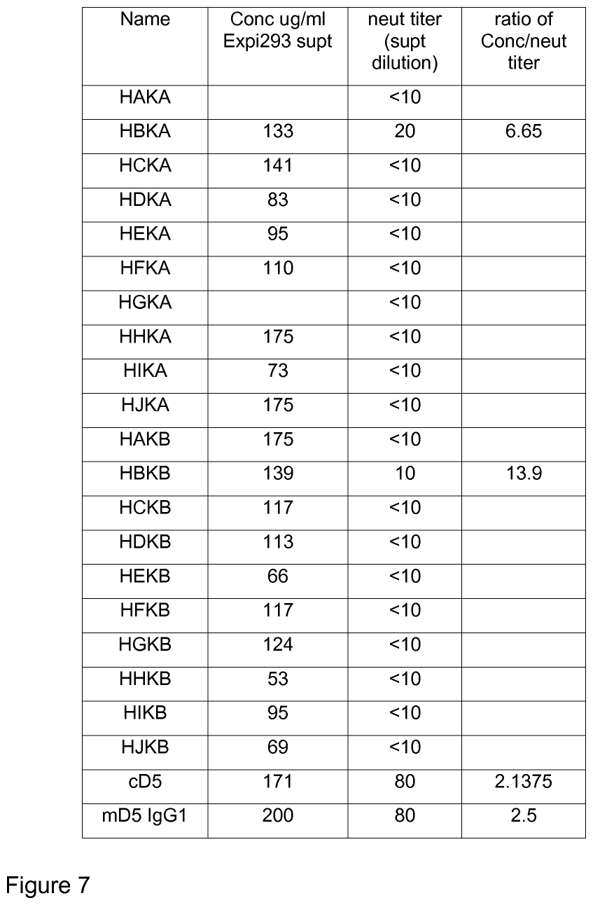

Humanized antibody heavy chain variants HA-HJ in combination with either KA or KB kappa light chains were tested for their neutralization capability. FIG. 7 shows that the antibody variants containing the HB heavy chain were the only variants capable of neutralizing the EV71 virus.

Often, in a humanized antibody, residues in the human framework within approximately 4A of the non-human CDR sequences (i.e. the 4A Proximity Residues) need to be replaced by the corresponding non-human residue to maintain antibody function. However, FIGS. 5, 6, and 7 show that backmutating the 4A Proximity Residues to murine residues in the humanized D5 antibody variants containing heavy chains HB-HJ does not achieve the expected maintenance of antibody function, and the binding capacity of the humanized D5 antibodies described in Example 3 is greatly reduced compared to that of the chimeric D5 antibody.

### Example 4. Efficacy of hEV71 HM-HQ Humanized Heavy Chains

In response to the unexpected loss of binding activity in the humanized antibody variants in Example 3, new versions of the humanized D5 heavy chain, containing new and/or different combinations of backmutations to murine residues, were generated to attempt to obtain humanized D5 antibodies with an improved binding capacity. Three promising heavy chain sequence variations were designated HM (SEQ ID NO:3), HN (SEQ ID NO:4), and HQ (SEQ ID NO:6).

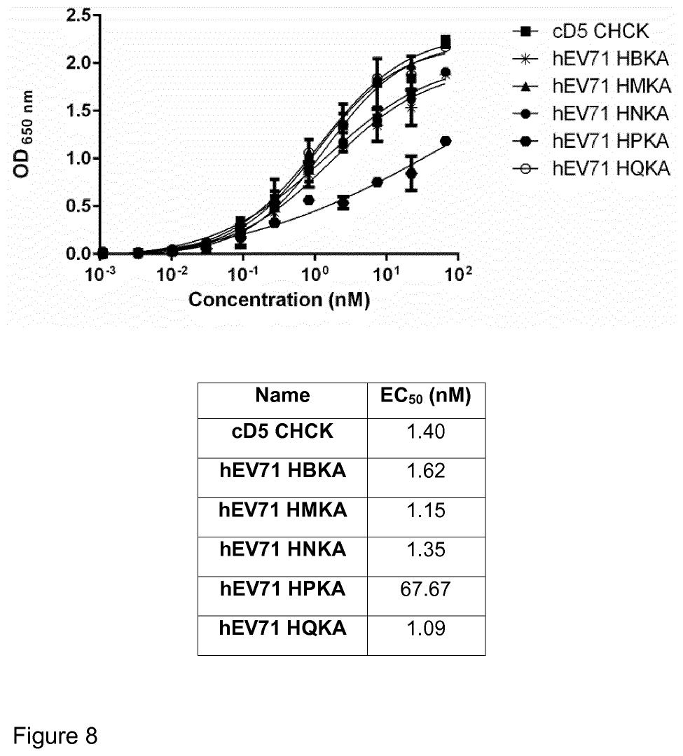

The HM-HQ heavy chains were combined with the KA version of the kappa light chain. The binding activities of hEV71 HMKA, hEV71 HNKA and hEV71 HQKA antibodies to the SP70 peptide epitope were measured by binding ELISA, shown in FIG. 8. The hEV71 HNKA version showed a binding capacity similar to that of the HBKA candidate. However, the hEV71 HMKA and hEV71 HQKA versions showed an unexpected improvement on binding to the SP70 peptide epitope, displaying a similar binding capacity to the chimeric D5 antibody. The two supplementary mutations present in the HM heavy chain (K23T and S77N in SEQ ID NO:3), in addition to the mutations already present in the HB heavy chain variant, improved the binding of the hEV71 HMKA humanized D5 antibody. The HQ heavy chain variant contains all the mutations present in the HB heavy chain, the two extra mutations in the HM heavy chain and an additional mutation: R38K. hEV71 HQKA does not appear to have a superior binding capacity over hEV71 HMKA, thus the R38K mutation does not seem to be crucial for antibody function.

One other humanized D5 heavy chain was generated, designated HP (SEQ ID NO:5). However, hEV71 HPKA showed poor binding to the SP70 peptide epitope (FIG. 8).

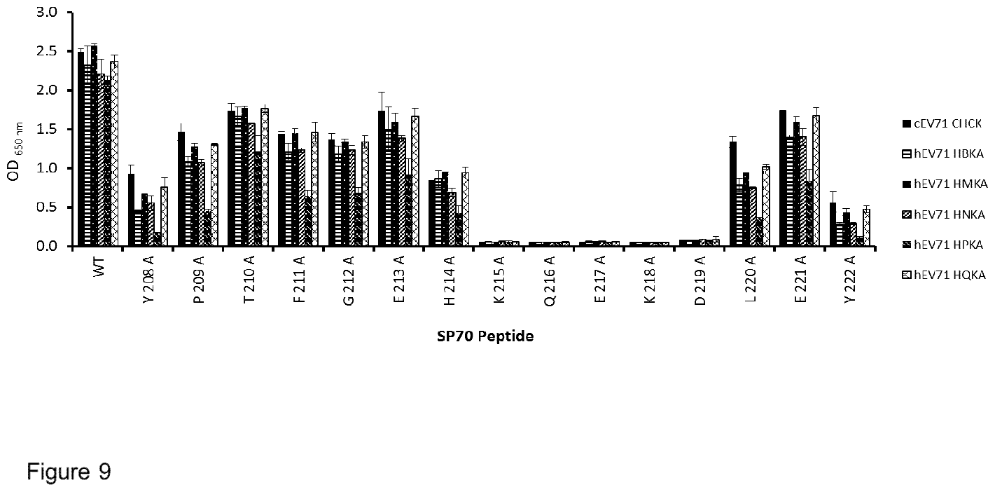

The binding of the hEV71 HMKA to hEV71 HQKA variants to 15 variations of the SP70 peptide epitope was tested by binding ELISA at the EC_{K}) of each antibody. The epitope variations were produced by mutating each amino acid in the SP70 peptide sequence (SEQ ID NO:12) to an alanine residue one at a time. The binding ELISA assay was performed as before. FIG. 9 shows that the HM to HQ antibody variants demonstrate the same pattern of binding to the SP70 peptide alanine scanning library as the chimeric D5 antibody and the HBKA variant. As observed in the binding ELISA in FIG. 8, hEV71 HMKA and hEV71 HQKA variants showed the highest binding capacity to the SP70 peptide epitope variations.

### Example 5. hEV71 HMKA Virus Neutralization

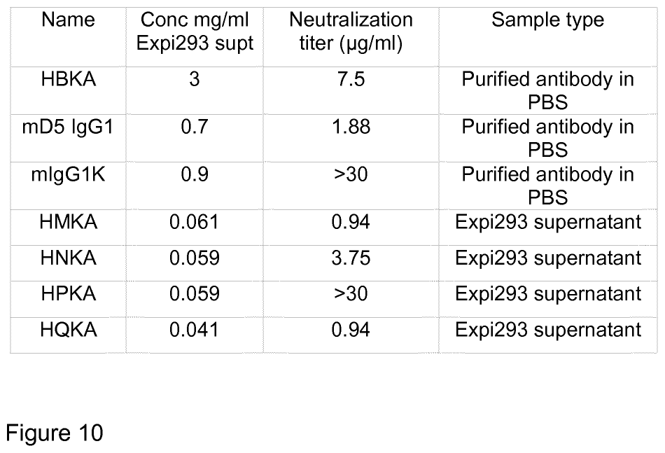

A neutralization assay was performed to assess the EV71 virus neutralization capability of the hEV71 HMKA to hEV71 HQKA humanized D5 antibody variants (FIG. 10). The hEV71 HNKA variant showed a small improvement on virus neutralization compared to hEV71 HBKA. hEV71 HPKA failed to neutralize the virus. However, hEV71 HMKA and hEV71 HQKA variants, and to a slightly lesser extent hEV71 HNKA, performed significantly better than hEV71 HBKA and showed an unexpected improvement compared to even the mouse D5 antibody.

### Example 6. hEV71 HMKA Antibody has a Similar Binding Affinity for SP70 Peptide as Chimeric D5 Antibody

This example demonstrates the observed binding constant (K_{A}) and the observed dissociation constant (K_{D}) of the hEV71 HMKA antibody. Kₐ describes how much [antibody][antigen] complex exists at the reaction point where equilibrium is reached with the rate of dissociation of the components into [antibody]+[antigen]. High affinity antibodies will bind a greater amount of antigen in a shorter period of time than low-affinity antibodies, and have a Kₐ value greater than 10⁷ M⁻¹. The lower the K_{d}, the greater the affinity of an antibody for its antigen or epitope. K_{d} values are usually in the low micromolar (10⁻⁶) to nanomolar (10⁻⁷ to 10⁻⁹) range for most antibodies, or in the low nanomolar (10⁻⁹) or even picomolar range (10⁻¹²) for high or very high affinity antibodies.

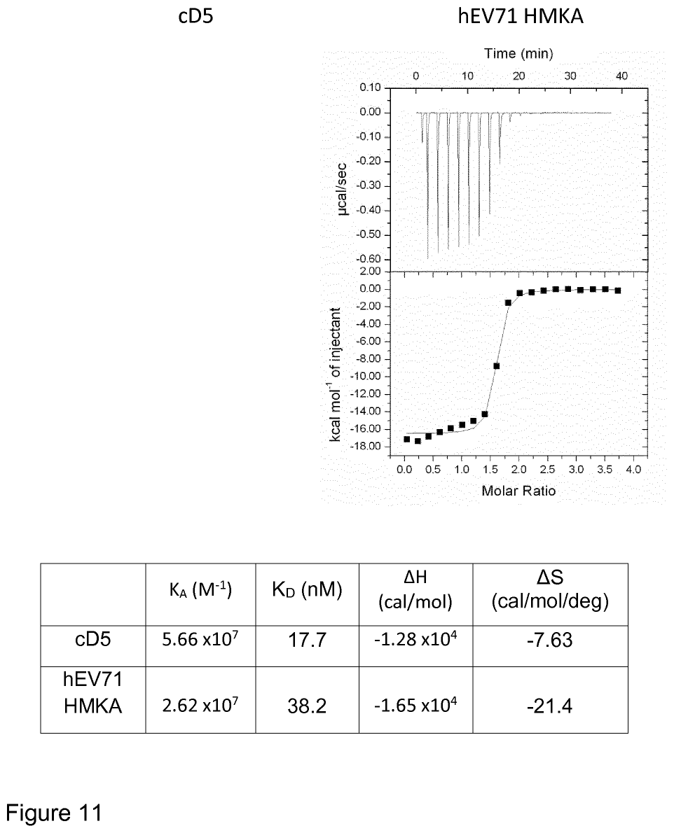

Binding of chimeric D5 and hEV71 HMKA antibodies to the SP70 peptide epitope was performed by isothermal titration calorimetry (ITC; FIG. 11), a thermodynamic technique which produces ‘observed’ K_{A} and K_{D} values. K_{D} can be calculated using Gibbs free energy, enthalpy and entropy: ΔG=ΔH−TΔS and ΔG=RT In K_{D}. K_{A} is calculated using K_{A}=1/K_{D}.

The hEV71 HMKA antibody bound to the SP70 peptide with an observed K_{A} of 2.62×10⁷ M⁻¹. Chimeric D5 antibody bound to the SP70 peptide with an observed K_{A} of 5.66×10⁷ M⁻¹. Both antibodies have a high affinity for the SP70 epitope.

FIG. 11 also reveals that the hEV71 HM KA antibody bound to the SP70 peptide with an observed K_{D} value of 38.2 nM (3.82×10⁻⁸ M), which is comparable to the chimeric D5 antibody observed K_{D} value of 17.1 nM (1.71×10⁻⁸ M), and is close to the general K_{D} range for a high affinity antibody.

Thus the hEV71 HM KA antibody has a high observed binding constant and a low observed dissociation constant for the SP70 peptide epitope on the VP1 capsid protein of the EV71 virus, similar to the values calculated for the chimeric D5 antibody. The murine mutations introduced into the framework regions of hEV71 HMKA have produced an antibody with a heavy chain that has the necessary murine D5 characteristics required for epitope binding. An antibody of the present invention can thus be used for clinical, e.g., therapeutic or prophylactic, use, or other uses, as described herein. In particular, an antibody of the present invention can be used for treatment of EV71 infection and/or HFMD and related diseases, or in a situation where an antibody of the present invention needs to bind to the SP70 peptide epitope of EV71.

The following examples provide a detailed biophysical analysis of the hEV71 HMKA antibody, discussing the properties of the antibody that are desirable or necessary in clinical reagents.

### Example 7. Thermal Stability of hEV71 HMKA

Monoclonal antibodies are relatively fragile and high temperatures can result in antibody denaturation, which creates proteins that are sensitive to irreversible aggregation. In addition, more stable antibodies have a longer half-life in patient serum, and can have beneficial effects on therapeutic dosing strategies.

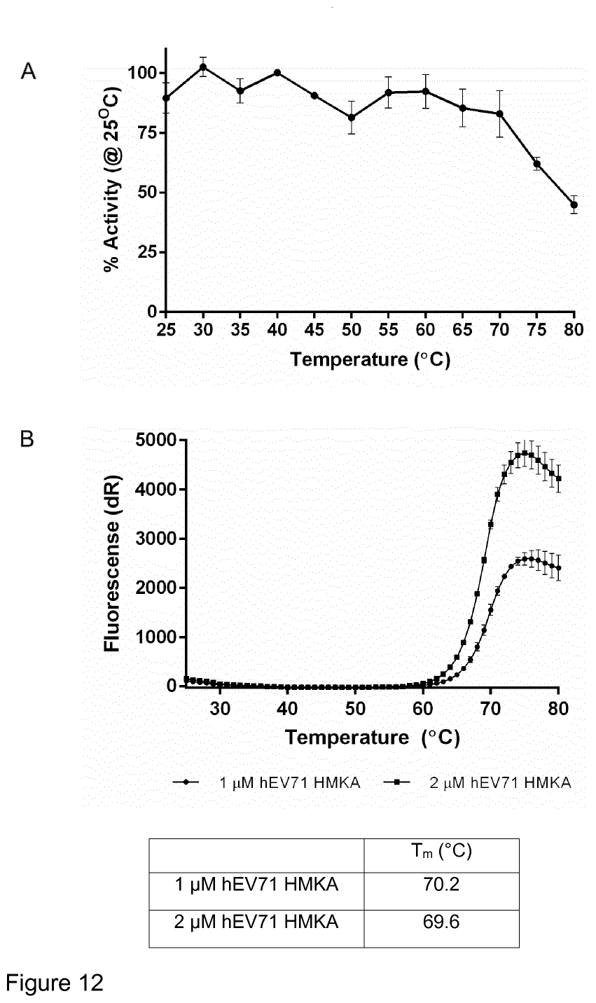

The thermal stability of hEV71 HMKA antibody was tested. hEV71 HMKA antibody was diluted to EC₈₀ concentration in 100 μl 1% milk/PBS/0.05% Tween 20. The antibody was subjected to 10 minute temperature treatments between 25° C. and 80° C. with 5° C. intervals, cooled to 4° C. and tested by binding ELISA. FIG. 12a shows that hEV71 HMKA appears stable, retaining its binding ability to the EV71 SP70 peptide epitope until 70° C., where binding to SP70 decreased.

The melting temperature (Tm) of hEV71 HMKA was tested in a thermal shift assay. Antibody at a final concentration of 1 or 2 μM was incubated with a fluorescent dye (Sypro Orange) for 71 cycles with 1° C. increase per cycle in a qPCR thermal cycler from 25° C. to 95° C. The Tm for hEV71 HMKA was calculated to be 69-70° C. (FIG. 12b).

A melting point of 69-70° C. and the ability to bind the SP70 epitope at temperatures up to and beyond its melting point demonstrates that antibodies of the present invention are suitable for therapeutic and prophylactic uses, are unlikely to denature in the body of a patient, and may have a long half-life so the patient may need fewer doses of an antibody to treat HFMD, related disease and/or EV71 infection.

### Example 8. Aggregation and Solubility of hEV71 HMKA

This example shows that antibodies of the present invention can be concentrated and remain in monomeric form.

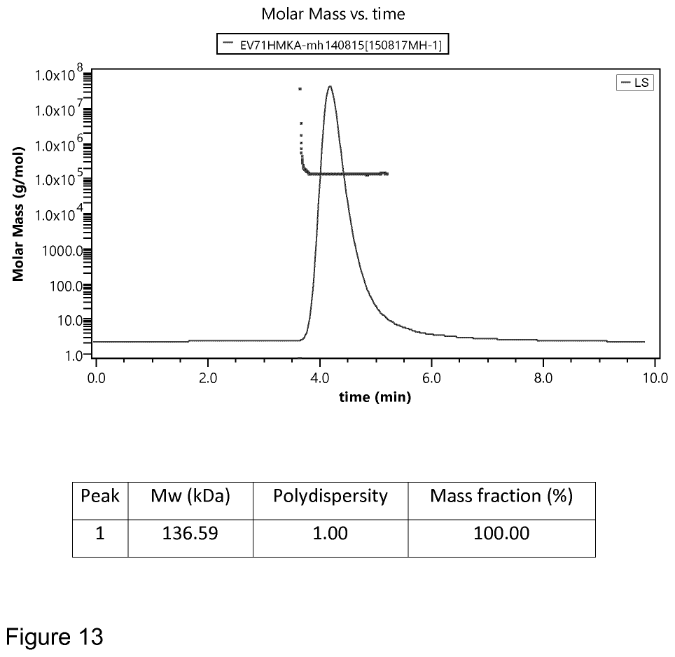

Samples of the hEV71 HMKA antibody were injected at 0.4 ml/min into a size exclusion column in an HPLC system and analyzed by multi-angle light scattering to determine the absolute molar masses and check for aggregation. The profile in FIG. 13 shows no signs of aggregation as the species detected had an average molecular mass of about 136.6 kDa, which is the expected range for an IgG monomer in this analysis. The antibody is monodispersed (Mw/Mn<1.05). The mass recovery is between 99.6-99.9% (calculated mass over injected mass), which indicates good protein recovery and that the sample does not seem to stick to the column or contain insoluble aggregates, which would be retained by the guard column. Overall the data suggest there are no aggregation concerns for the hEV71 HMKA antibody.

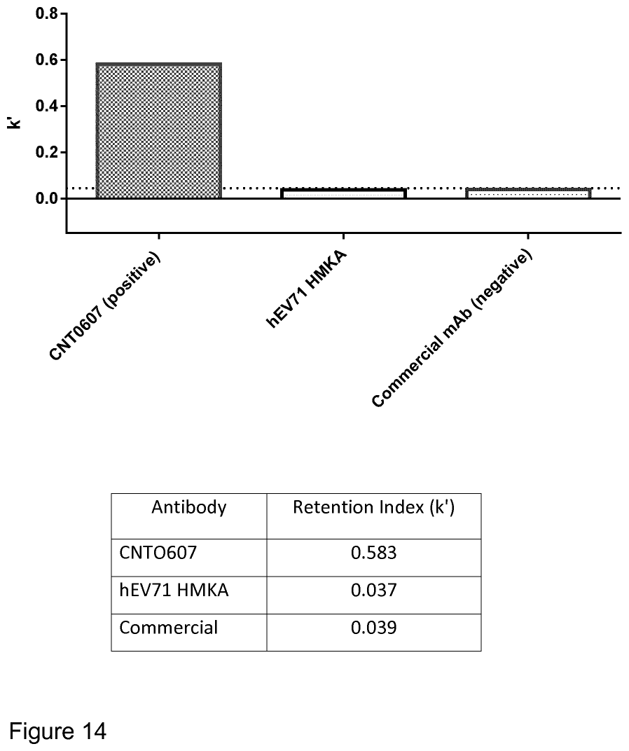

Cross-Interaction Chromatography (CIC) analysis was performed to assess the propensity of an antibody to become involved with non-specific protein-protein interactions and to provide an indication of any solubility issues, which can give rise to downstream manufacturing problems. CIC can be used to discriminate between soluble and insoluble antibodies. An elevated Retention Index (k′) indicates a self-interaction propensity and a low solubility. Samples were analyzed by two separate 20 μl injections, one onto a 1 ml NHS activated resin with 30 mg of human polyclonal IgG, and the other onto a 1 ml NHS activated control column. Eluted samples were detected by UV absorbance. Sample peak retention times were used to calculate the retention factor k′. FIG. 14 illustrates that the hEV71 HM KA antibody shows a Retention Index below 0.037, indicating a low propensity for non-specific interactions and good solubility.

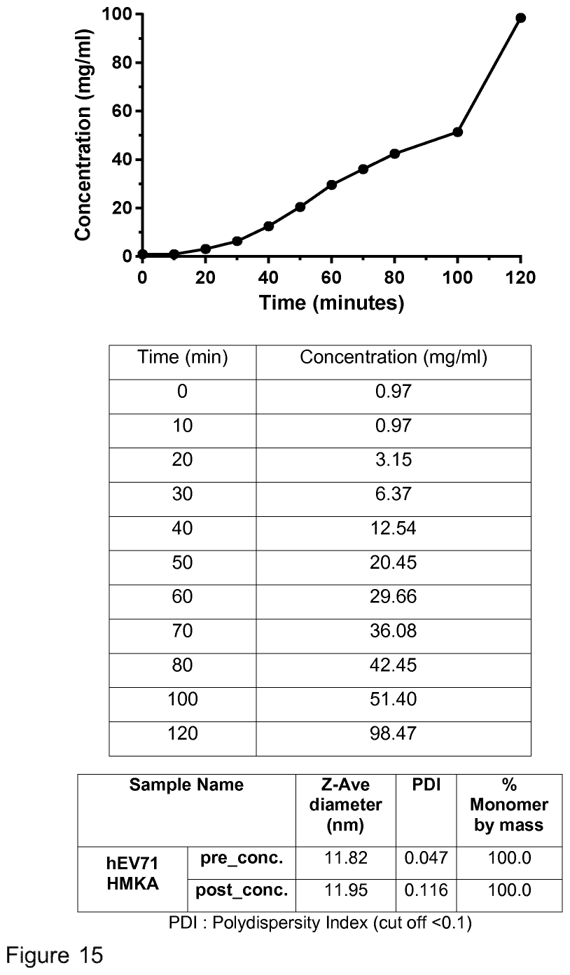

Dynamic light scattering (DLS) was used as a complementary technique (i.e., to static-light scattering such as SEC-MALS) for the detection of soluble aggregates. Triplicate 30 ml samples of hEV71 HM KA antibody in Dulbecco's PBS (Sigma; 2.7 mM KCl, 1.5 mM KH₂PO₄, 138.0 mM NaCl, 8.1 mM Na₂HPO₄; physiological pH) were concentrated using solvent absorption concentrators (MWCO 7500 kDa) and the concentration measured at timed intervals (FIG. 15). The antibody was concentrated to 98.5 mg/ml without apparent precipitation. Aggregation assessment DLS showed that, after concentration, the antibodies were found to have hydrodynamic radii and polydispersity consistent with monomer. The data suggest that the antibody is not prone to precipitation at concentrations of up to 98 mg/ml. The mean particle size (Z-Ave diameter) and the polydispersity index (PDI) were obtained by cumulants analysis and show similar values for the pre-concentrated and post-concentrated antibody samples.

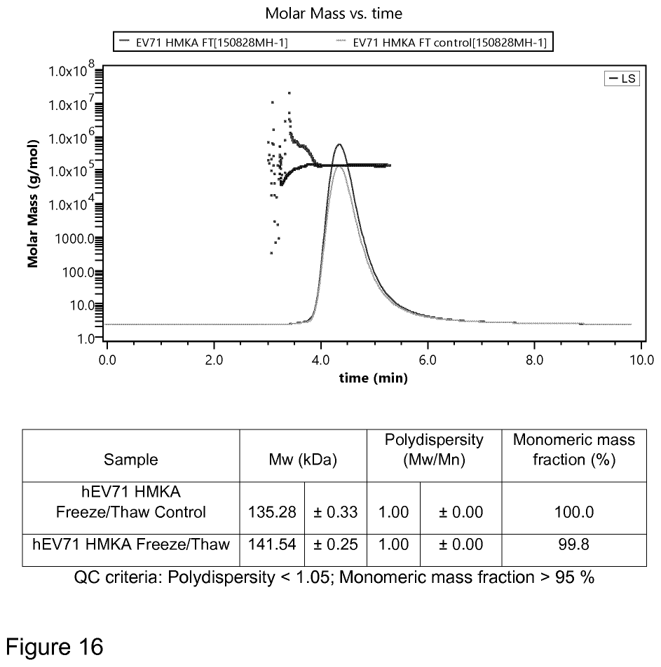

The hEV71 HMKA antibody was further tested for aggregation propensity during freeze/thaw stress. Samples of the purified candidate antibodies were subjected to 10 cycles of 15 minutes at −80° C. followed by thawing for 15 minutes at room temperature. ‘Non-stressed’ and ‘stressed’ samples were then analyzed by SEC-MALS (size exclusion chromatography—multi angle light scattering) to check for the presence and/or induction of aggregates, which could give rise to downstream manufacturing issues. The data in FIG. 16 suggests that freeze/thaw does not cause aggregation in the hEV71 HMKA antibody. The molecular weight of hEV71 HMKA after stress is about 141.5 kDa, near to the monomeric weight of an IgG. The antibody is monodispersed (Mw/Mn<1.05), meaning that only one molecular weight is present, and the monomeric mass fraction is 99.8%.

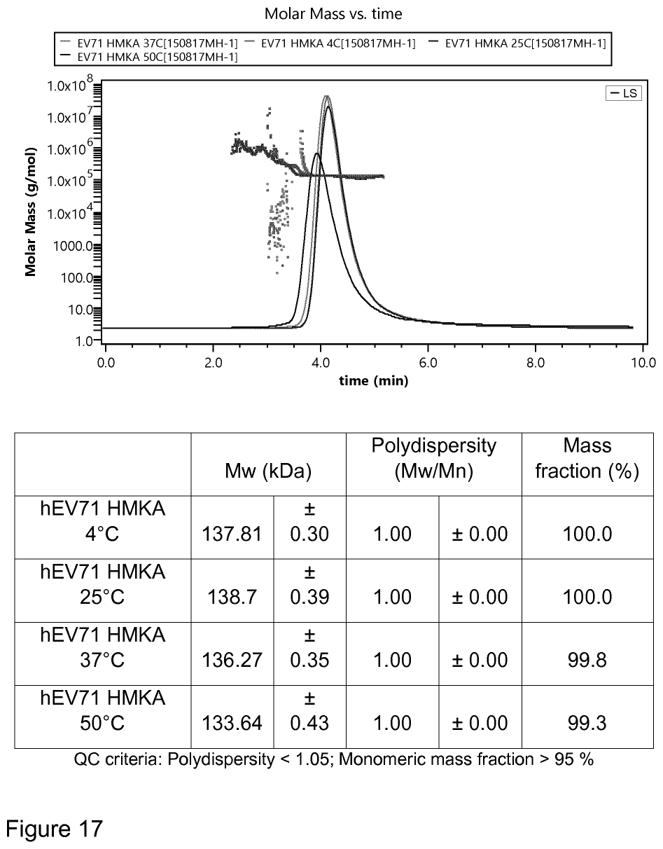

hEV71 HM KA antibody was subjected to heat-induced stress analysis to investigate aggregation. Samples of the purified candidate antibodies were exposed at a) 4° C., b) 25° C., c) 37° C. and d) 50° C. for 33 days. Samples were then analyzed by SEC-MALS as above to check for aggregation. FIG. 17 shows that the molecular weight of hEV71 HMKA remains around the monomeric weight of 136 kDa at all four temperatures. The antibody is monodispersed (Mw/Mn<1.05), meaning that only one molecular weight is present, and the mass fraction value was not affected by heat treatment.

The isoelectric point (pl) of the hEV71 HMKA antibody was determined by capillary isoelectric focusing (cIEF), a method that allows separation of proteins based on their pl through a pH gradient by the application of voltage across a capillary field, where the opposing ends are submerged in acidic (anodic) and basic (cathodic) solutions. Proteins stop migrating once they reach their isoelectric point and possess a neutral charge. The technique was performed with a 5-10 mg/ml protein solution containing ≤50 mM NaCl, in the following buffers: anolyte (200 mM Phosphoric Acid), catholyte (300 mM Sodium Hydroxide), chemical mobilizer (350 mM Acetic Acid), cathodic stabilizer (500 mM Arginine), anodic stabilizer (200 mM Iminodiacetic Acid), 4.3M urea solution, and 3M urea—clEF gel solution. The pl of the main peak for the hEV71 HMKA antibody was determined to be at pH 7.4. Overall the data suggest that the hEV71 HMKA antibody has excellent biophysical properties and there are no aggregation or solubility concerns. The hEV71 HMKA antibody remained in a monomeric state, shows a low propensity for non-specific interactions, does not appear to have any solubility issues, does not aggregate or precipitate when concentrated, does not undergo aggregation when subjected to repeated cycles of freezing and thawing, and does not show a propensity to aggregate when subjected to elongated periods of heat treatment. The hEV71 HM KA antibody passes all QC criteria for biophysical properties. In combination, these data demonstrate that an antibody of the present invention can be manufactured, stored and used, as described herein, without becoming damaged or unusable by such treatment.

### Example 9. Serum Stability Assessment of hEV71 HMKA

This example demonstrates the stability of the hEV71 antibody in mammalian serum.

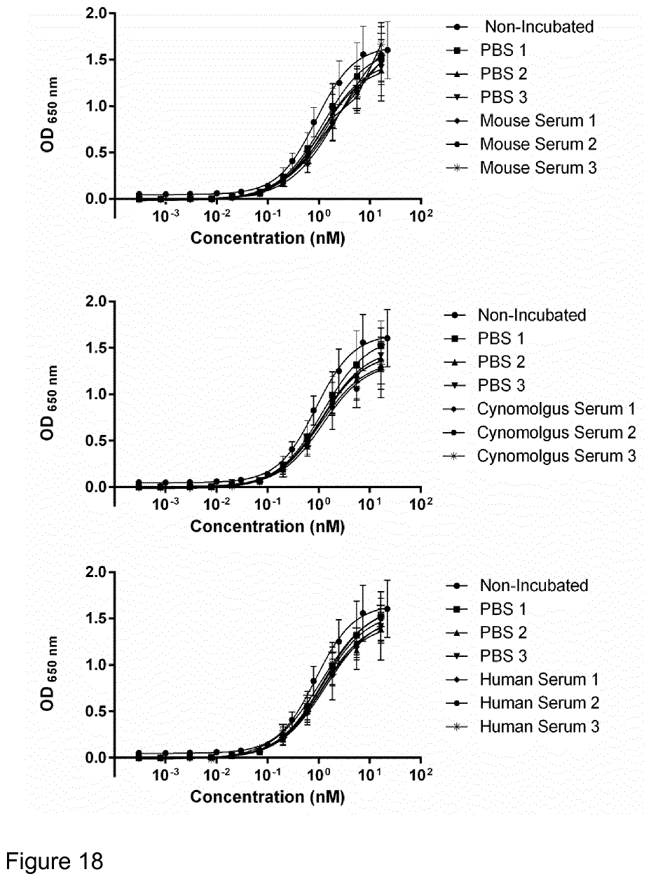

Purified samples of hEV71 HMKA antibody in PBS were incubated in mouse, human and cynomolgus serum for 5, 10, 20 and 30 days at 37° C. The binding affinity of the antibody after the incubation was measured by binding ELISA to the SP70 peptide epitope. The binding of hEV71 HM KA which had been incubated in the 3 different serums was compared with antibody binding which had not undergone any incubation and antibody which had been incubated in PBS. The ELISA assays in FIG. 18 show that the binding of the serum incubated antibody to the SP70 peptide epitope is very similar to the binding of the PBS incubated and non-incubated antibody after 31 days. Therefore incubation of the hEV71 HMKA antibody in mouse, human and cynomolgus serum for a month does not have an effect on the binding function of the antibody to the SP70 peptide epitope.

Serum stability of an antibody is important if the antibody is for use in subjects or in cells of such subjects. Antibodies of the present invention retain binding capacity for the SP70 epitope when in a serum-based environment, and are therefore useful for therapeutic and prophylactic treatments.

### Example 10. Crucial Residues for Antibody Function

The structure of the fab region of the chimeric D5 antibody whilst in complex with the SP70 peptide was resolved by X-ray crystallography. Analysis of the structure revealed that the side chain of asparagine residue number 76 on the mouse D5 heavy chain (numbering according to Kabat; equivalent to back-mutated residue N77 in SEQ ID NO:3) forms hydrogen bonds to the backbone of phenylalanine 27 and asparagine 28. These two residues are positioned 2 amino acids before the start of the CDR loop 1 of the variable heavy chain and they appear to be necessary for the stabilization of this loop for antigen recognition. It is therefore key for this asparagine residue (N76 (Kabat)) to be retained to preserve this function.

hEV71 HMKA and hEV71 HQKA humanized antibody variants (SEQ ID NO:3 and SEQ ID NO:6 respectively) contain the back-mutated murine D5 N77 residue in addition to the D5 F27 and N28 residues. This provides an explanation for the higher binding affinity and virus neutralization capacity of HMKA and HQKA variants when compared to antibodies containing the heavy chain variant HB mutations (SEQ ID NO:11) which contains a human serine residue at position 77, rather than asparagine.

### Example 11. In Vivo Efficacy of hEV71 HMKA

Treatment with hEV71 HMKA results in a 100% survival rate of mice infected with EV71.

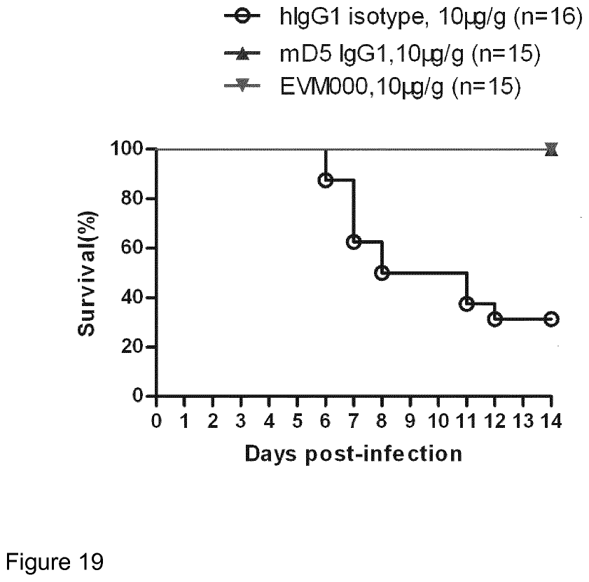

For therapeutic studies, groups of seven-day-old ICR mice were i.p. inoculated with 5×10⁵ TCI D₅₀ of EV71/MAV-W. Then the mice were administered a single dose (10 ug/g body weight) of mAb (mouse D5 (n=15), hEV71 HMKA (EVMOO; n=15), or human IgG1 isotype control (n=16)) by i.p. injection at a 1 day after infection. All mice were monitored daily for survival and clinical signs for a period of 14 days. Clinical scores were graded as follows: 0, healthy; 1, reduced mobility; 2, limb weakness; 3, paralysis; 4, death. As shown in FIG. 19, the mice receiving the control antibody eventually developed severe disease and died (with a mortality rate of ˜67%); in contrast, all of the mice that had received either mouse D5 or humanized D5 (hEV71 HMKA; EVM00) survived without severe clinical signs. These results indicate that an antibody of the present invention fully retains therapeutic capacity and acts to neutralize the EV71 virus in vivo, and can thus be used for therapeutic and prophylactic uses as described herein.

### Example 12. hEV71 HMKA Antibody Buffer Scouting

Buffer scouting was carried out for the hEV71 HM KA antibody by measurement of size distribution using the dynamic light scattering method on a Malvern Zetasizer APS instrument. An antibody concentration of 0.5 mg/ml was used for all measurements.

Hydrodynamic radius of the hEV71 HMKA antibody was measured in buffers shown in Table 2 below by taking three single measurements at room temperature. At room temperature, hEV71 HM KA antibody remained soluble in the sodium acetate, sodium citrate, sodium phosphate, and sodium succinate buffers to a degree comparable with the PBS control (Table 3). No aggregates were detected.

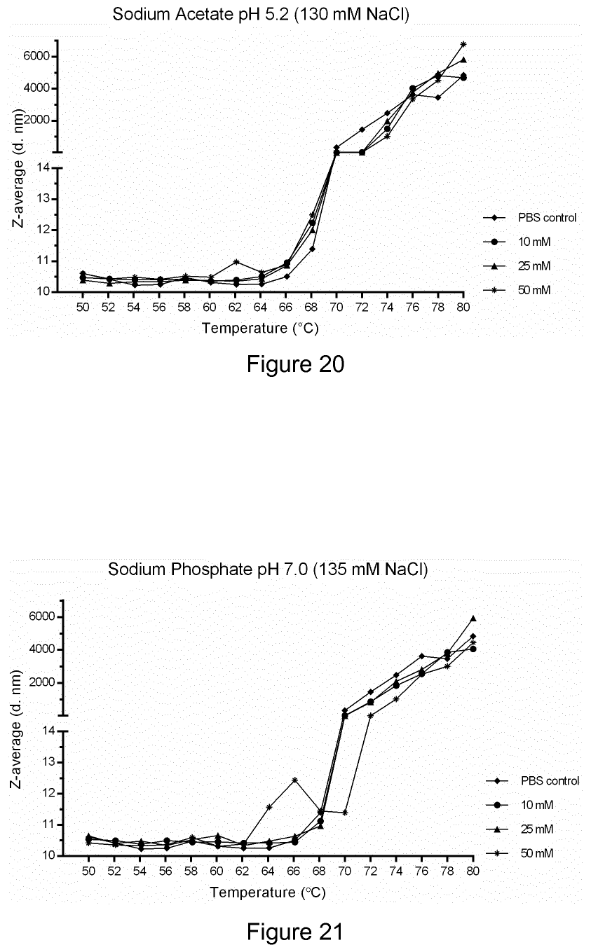

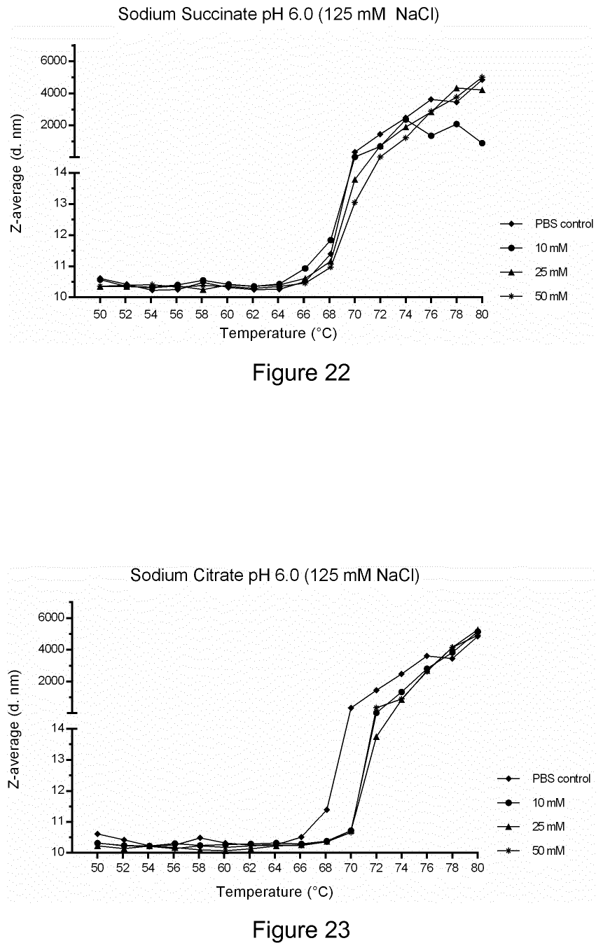

A temperature ramp was performed in the same buffers to determine the temperature at which the antibody starts to aggregate. One measurement was taken per temperature at 2° C. increments from 50° C. to 80° C. hEV71 HMKA antibody was shown to aggregate at around 70° C. in PBS buffer and to have a similar aggregation temperature of 70° C. in the 10, 25 and 50 mM Sodium Acetate 130 mM NaCl pH 5.2 buffers (FIG. 20), in 10, 25 and 50 mM Sodium Phosphate 135 mM NaCl pH 7.0 buffers (FIG. 21), and in 10, 25 and 50 mM Sodium Succinate 125 mM NaCl pH 6.0 buffers (FIG. 22). In the 10, 25 and 50 mM Sodium Citrate 125 mM NaCl pH 6.0 buffers, the aggregation temperature increased to around 72° C. (FIG. 23).

These results indicate that a sodium acetate, sodium phosphate, sodium succinate and/or sodium citrate formulation can be used for the hEV71 HMKA antibody, and that the sodium citrate buffer formulation increases the stability of the hEV71 HMKA antibody.

TABLE 2
Buffer Solutions
Buffer

10 mM Sodium Acetate, 130 mM NaCl pH 5.2
25 mM Sodium Acetate, 130 mM NaCl pH 5.2
50 mM Sodium Acetate, 130 mM NaCl pH 5.2
10 mM Sodium Citrate, 125 mM NaCl pH 6.0
25 mM Sodium Citrate, 125 mM NaCl pH 6.0
50 mM Sodium Citrate, 125 mM NaCl pH 6.0
10 mM Sodium Succinate, 125 mM NaCl pH 6.0
25 mM Sodium Succinate, 125 mM NaCl pH 6.0
50 mM Sodium Succinate, 125 mM NaCl pH 6.0
10 mM Sodium Phosphate, 135 mM NaCl pH 7.0
25 mM Sodium Phosphate, 135 mM NaCl pH 7.0
50 mM Sodium Phosphate, 135 mM NaCl pH 7.0

TABLE 3
Antibody solubility assay

| | Z-average |
|---|---|
| | (d · nm) |
| PBS | 10.4 |
| 10 mM Sodium Acetate, 130 mM NaCl pH 5.2 | 10.4 |
| 25 mM Sodium Acetate, 130 mM NaCl pH 5.2 | 10.4 |
| 50 mM Sodium Acetate, 130 mM NaCl pH 5.2 | 10.5 |
| PBS | 10.3 |
| 10 mM Sodium Citrate, 125 mM NaCl pH 6.0 | 10.3 |
| 25 mM Sodium Citrate, 125 mM NaCl pH 6.0 | 10.2 |
| 50 mM Sodium Citrate, 125 mM NaCl pH 6.0 | 10.2 |
| PBS | 10.4 |
| 10 mM Sodium Phosphate, 135 mM NaCl pH 7.0 | 10.5 |
| 25 mM Sodium Phosphate, 135 mM NaCl pH 7.0 | 10.7 |
| 50 mM Sodium Phosphate, 135 mM NaCl pH 7.0 | 10.4 |
| PBS | 10.4 |
| 10 mM Sodium Succinate, 125 mM NaCl pH 6.0 | 10.4 |
| 25 mM Sodium Succinate, 125 mM NaCl pH 6.0 | 10.4 |
| 50 mM Sodium Succinate, 125 mM NaCl pH 6.0 | 10.4 |

(Z-average = the intensity weighted mean hydrodynamic size in diameters; expected diameter for IgG is around 10 nm.)

### Example 13. Protective Effect of hEV71 HMKA in Infant Monkeys Infected by EV71

Groups of 1.5±0.5 month old rhesus monkeys were infected with 1×10⁶ CCID₅₀ EV71 by tracheal instillation. The monkeys were administered a single intravenous dose of hEV71 HMKA (“Antibody Treatment”, 5 mg/kg body weight, n=4), control human IgG1k antibody (“hIgG1K Control”, 5 mg/kg body weight, n=3), or an empty solvent control (“Model Control”, 0.9% w/v NaCl, n=2) on the same day as infection. All monkeys were monitored for 14 days post-infection. Changes in body temperature and blood viral load were recorded.

After the EV71 virus infection, the solvent control group showed a significant increase in body temperature and increased viral load in blood consistent with the clinical manifestations of HFMD.

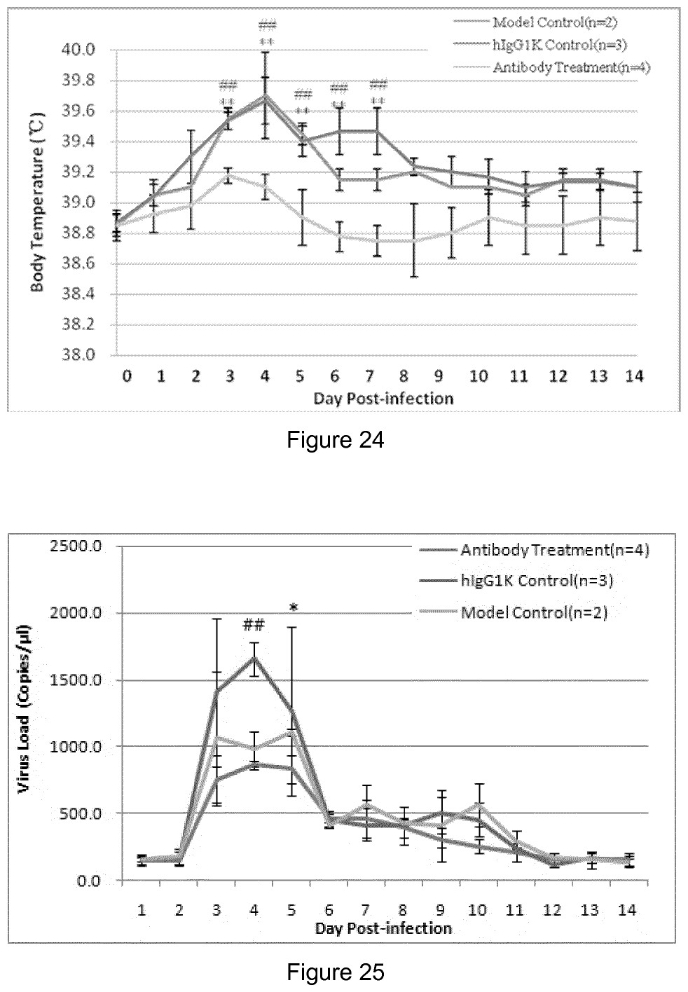

The animals of the solvent treated group and the control antibody treated group showed a significant increase in body temperature after infection (FIG. 24, Table 4). The animals in the group treated with hEV71 HMKA showed a slight increase in body temperature after infection, but the increase was less than that of the other two groups. Three to seven days after EV71 infection, the body temperature of the group treated with huEV71 HMKA was significantly lower than that of the other two groups.

Blood viral load increased after infection in each group. On day 4 to day 5 after EV71 infection, the viral loads in the blood samples of the antibody treatment group were significantly lower than those of the other two groups (FIG. 25, Table 5).

The observed reduced body temperature and viral load of antibody treated animals relative to the control groups indicates that huEV71 HM KA has the effect of treating EV71 virus infection in this in vivo model.

TABLE 4
Body temperature after infection.

| | Test Groups (° C., Mean ± SD) | | |
|---|---|---|---|
| Days post- | Model Control | Control Antibody | EV71 antibody |
| infection | (n = 2) | (n = 3) | (n = 4) |
| 0 | 38.9 ± 0.1 | 38.9 ± 0.1 | 38.9 ± 0.1 |
| 1 | 39.1 ± 0.1 | 39.0 ± 0.1 | 38.9 ± 0.1 |
| 2 | 39.1 ± 0.0 | 39.3 ± 0.2 | 39.0 ± 0.2 |
| 3 | 39.6 ± 0.1 | 39.5 ± 0.1 | 39.2 ± 0.1**## |
| 4 | 39.7 ± 0.3 | 39.7 ± 0.2 | 39.1 ± 0.1**## |
| 5 | 39.5 ± 0.1 | 39.4 ± 0.1 | 38.9 ± 0.2**## |
| 6 | 39.2 ± 0.1 | 39.5 ± 0.2 | 38.8 ± 0.1**## |
| 7 | 39.2 ± 0.1 | 39.5 ± 0.2 | 38.8 ± 0.1**## |
| 8 | 39.2 ± 0.0 | 39.2 ± 0.1 | 38.8 ± 0.2 |
| 9 | 39.1 ± 0.0 | 39.2 ± 0.1 | 38.8 ± 0.2 |
| 10 | 39.1 ± 0.0 | 39.2 ± 0.1 | 38.9 ± 0.2 |
| 11 | 39.1 ± 0.1 | 39.1 ± 0.1 | 38.9 ± 0.2 |
| 12 | 39.2 ± 0.1 | 39.1 ± 0.1 | 38.9 ± 0.2 |
| 13 | 39.2 ± 0.1 | 39.1 ± 0.1 | 38.9 ± 0.2 |
| 14 | 39.1 ± 0.0 | 39.1 ± 0.1 | 38.9 ± 0.2 |

Where compared with the model control group, *p < 0.05, **p < 0.01, and compared with the control antibody group, #p < 0.05, ##p < 0.01.

TABLE 5
Blood viral load in the blood of animals after infection

| | Test Groups (Copies/μl, Mean ± SD) | | |
|---|---|---|---|
| Days post- | Model Control | Control Antibody | EV71 antibody |
| infection | (n = 2) | (n = 3) | (n = 4) |
| 1 | 157.2 ± 16.7 | 157.1 ± 36.8 | 146.1 ± 33.4 |
| 2 | 177.0 ± 60.4 | 170.8 ± 45.4 | 146.8 ± 33.0 |
| 3 | 1067.4 ± 491.6 | 1408.1 ± 553.3 | 746.9 ± 189.4 |
| 4 | 980.8 ± 133.5 | 1658.7 ± 123.6 | 864.2 ± 33.4## |
| 5 | 1106.6 ± 27.0 | 1265.2 ± 628.8 | 832.4 ± 106.7* |
| 6 | 409.8 ± 20.0 | 451.6 ± 45.2 | 455.1 ± 60.2 |
| 7 | 569.6 ± 149.7 | 413.8 ± 119.7 | 459.4 ± 142.6 |
| 8 | 429.6 ± 28.3 | 408.7 ± 137.4 | 390.8 ± 77.0 |
| 9 | 407.7 ± 267.0 | 504.0 ± 115.4 | 298.6 ± 53.3 |
| 10 | 559.8 ± 162.2 | 454.9 ± 128.8 | 253.6 ± 50.3 |
| 11 | 291.6 ± 80.9 | 244.0 ± 4.3 | 209.1 ± 64.6 |
| 12 | 166.2 ± 37.3 | 118.3 ± 15.1 | 153.9 ± 53.7 |
| 13 | 153.6 ± 60.7 | 167.9 ± 32.6 | 153.2 ± 24.2 |
| 14 | 137.0 ± 28.4 | 143.7 ± 38.6 | 151.4 ± 49.0 |

Where compared with the model control group, *p < 0.05, **p < 0.01; and compared with the control antibody group, #p < 0.05, ##p < 0.01.

Sequence listing

| | | |
|---|---|---|
| SEQ ID NO: 1 | HA | QVQLVQSGAEVKKPGASVKVSCKASGYTFTDTYIHWVRQAP |
| | | GQRLEWIGKIDPANGNTKYDPKFQDRVTITRDTSASTAYMEL |
| | | SSLRSEDTAVYYCASSNYWFDFDYWGQGTLVTVSS |
| SEQ ID NO: 2 | KA | DIVMTQSPLSLPVTPGEPASISCRSSQSIVHSNGNTYLEWYL |
| | | QKPGQSPQLLIYKVSNRFSGVPDRFSGSGSGTDFTLKISRVE |
| | | AEDVGVYYCYQGSHVPYTFGGGTKVEIK |
| SEQ ID NO : 3 | HM | 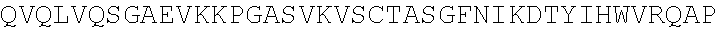 |
| | | 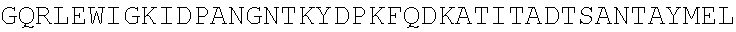 |
| | | 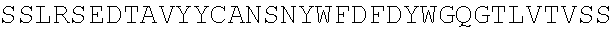 |
| SEQ ID NO: 4 | HN | 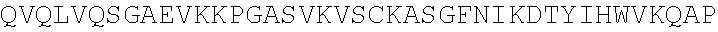 |
| | | 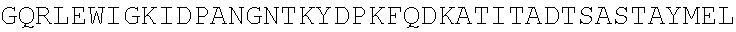 |
| | | 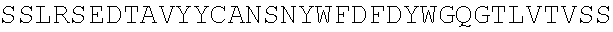 |
| SEQ ID NO: 5 | HP | QVQLVQSGAEVKKPGASVKVSCKASGYTFTDTYIHWVRQAP |
| | | 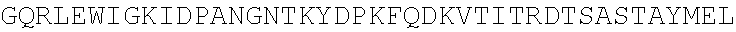 |
| | | 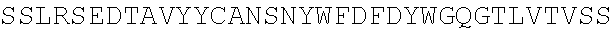 |
| SEQ ID NO: 6 | HQ | 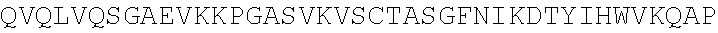 |
| | | 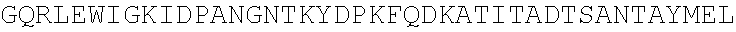 |
| | | 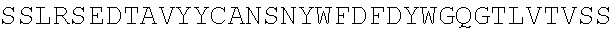 |
| SEQ ID NO: 7 | KB | 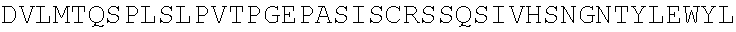 |
| | | QKPGQSPQLLIYKVSNRFSGVPDRFSGSGSGTDFTLKISRVE |
| | | AEDVGVYYCYQGSHVPYTFGGGTKVEIK |
| SEQ ID NO: 8 | KC | 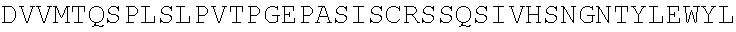 |
| | | QKPGQSPQLLIYKVSNRFSGVPDRFSGSGSGTDFTLKISRVE |
| | | AEDVGVYYCYQGSHVPYTFGGGTKVEIK |
| SEQ ID NO: 9 | KD | 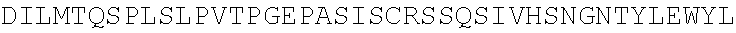 |
| | | QKPGQSPQLLIYKVSNRFSGVPDRFSGSGSGTDFTLKISRVE |
| | | AEDVGVYYCYQGSHVPYTFGGGTKVEIK |
| SEQ ID NO: 10 | KE | 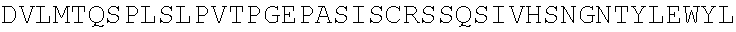 |
| | | 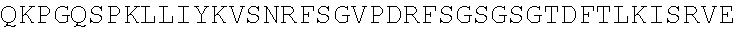 |
| | | AEDVGVYYCYQGSHVPYTFGGGTKVEIK |
| SEQ ID NO: 11 | HB | 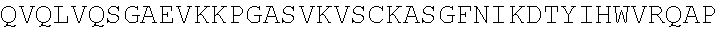 |
| | | 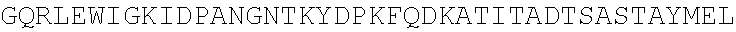 |
| | | 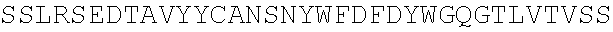 |
| SEQ ID NO: 12 | SP70 | YPTFGEHKQEKDLEY |
| | epitope | |
| SEQ ID NO: 13 | HC | 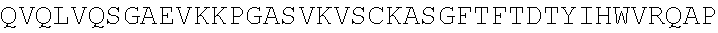 |
| | | GQRLEWIGKIDPANGNTKYDPKFQDRVTITRDTSASTAYMEL |
| | | SSLRSEDTAVYYCASSNYWFDFDYWGQGTLVTVSS |
| SEQ ID NO: 14 | HD | 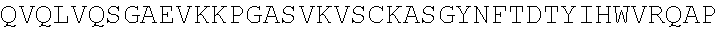 |
| | | GQRLEWIGKIDPANGNTKYDPKFQDRVTITRDTSASTAYMEL |
| | | SSLRSEDTAVYYCASSNYWFDFDYWGQGTLVTVSS |
| SEQ ID NO: 15 | HE | 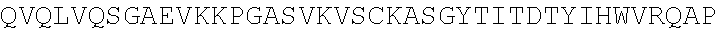 |
| | | GQRLEWIGKIDPANGNTKYDPKFQDRVTITRDTSASTAYMEL |
| | | SSLRSEDTAVYYCASSNYWFDFDYWGQGTLVTVSS |
| SEQ ID NO: 16 | HF | 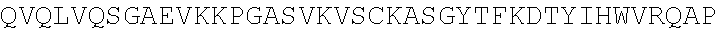 |
| | | GQRLEWIGKIDPANGNTKYDPKFQDRVTITRDTSASTAYMEL |
| | | SSLRSEDTAVYYCASSNYWFDFDYWGQGTLVTVSS |
| SEQ ID NO: 17 | HG | QVQLVQSGAEVKKPGASVKVSCKASGYTFTDTYIHWVRQAP |
| | | 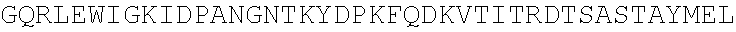 |
| | | SSLRSEDTAVYYCASSNYWFDFDYWGQGTLVTVSS |
| SEQ ID NO: 18 | HH | QVQLVQSGAEVKKPGASVKVSCKASGYTFTDTYIHWVRQAP |
| | | 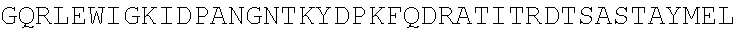 |
| | | SSLRSEDTAVYYCASSNYWFDFDYWGQGTLVTVSS |
| SEQ ID NO: 19 | HI | QVQLVQSGAEVKKPGASVKVSCKASGYTFTDTYIHWVRQAP |
| | | 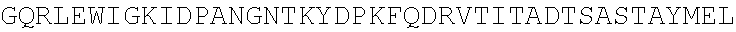 |
| | | SSLRSEDTAVYYCASSNYWFDFDYWGQGTLVTVSS |
| SEQ ID NO: 20 | HJ | QVQLVQSGAEVKKPGASVKVSCKASGYTFTDTYIHWVRQAP |
| | | GQRLEWIGKIDPANGNTKYDPKFQDRVTITRDTSASTAYMEL |
| | | 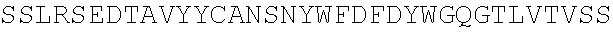 |
| SEQ ID NO: 21 | HA | CAGGTGCAGCTGGTCCAGTCAGGAGCAGAAGTCAAAAAG |
| | | CCCGGAGCATCAGTCAAAGTCTCTTGTAAAGCCTCAGGAT |
| | | ACACATTCACCGACACATACATCCACTGGGTGAGGCAGG |
| | | CTCCAGGACAGCGACTGGAGTGGATCGGCAAGATTGATC |
| | | CCGCCAACGGGAATACTAAGTACGACCCTAAATTCCAGGA |
| | | TCGGGTGACTATTACCAGAGACACCTCCGCCTCTACAGCT |
| | | TATATGGAGCTGAGCTCCCTGCGAAGCGAAGATACAGCA |
| | | GTCTACTATTGCGCCTCTAGTAACTACTGGTTCGACTTTGA |
| | | TTATTGGGGCCAGGGGACACTGGTGACTGTCTCAAGC |
| SEQ ID NO: 22 | HM | CAGGTCCAGCTGGTGCAGAGCGGAGCCGAAGTCAAAAAG |
| | | CCCGGAGCCTCAGTCAAAGTGTCTTGTAccGCATCAGGAT |
| | | TCAACATCAAAGACACCTACATCCACTGGGTGCGGCAGG |
| | | CACCAGGACAGAGACTGGAGTGGATCGGCAAGATTGATC |
| | | CCGCTAACGGGAATACAAAGTACGACCCTAAATTCCAGGA |
| | | TAAGGCAACCATTACAGCCGACACATCAGCCAaCACTGCT |
| | | TATATGGAGCTGAGCTCCCTGAGGTCCGAAGATACCGCT |
| | | GTCTACTATTGCGCAAACAGTAATTACTGGTTCGACTTTGA |
| | | TTATTGGGGCCAGGGGACTCTGGTGACCGTCTCTAGT |
| SEQ ID NO: 23 | KA | GACATTGTGATGACCCAGTCCCCTCTGAGCCTGCCCGTG |
| | | ACCCCCGGCGAACCTGCCTCCATTTCCTGCCGATCCAGC |
| | | CAGTCCATTGTGCACAGCAACGGAAATACATACCTGGAGT |
| | | GGTATCTGCAGAAGCCCGGCCAGTCCCCTCAGCTGCTGA |
| | | TCTACAAAGTGAGTAACCGGTTCTCAGGAGTCCCAGACCG |
| | | GTTCAGCGGCTCCGGGTCTGGAACCGATTTCACACTGAA |
| | | GATTTCTAGGGTGGAGGCCGAAGACGTGGGCGTCTACTA |
| | | TTGCTACCAGGGGAGCCATGTGCCCTATACTTTTGGCGG |
| | | GGGAACCAAGGTCGAAATCAAA |

1. An antibody comprising a heavy chain variable domain and a light chain variable domain, wherein
a) the heavy chain variable domain (VH domain) comprises SEQ ID NO:1 having at least 6 of the following 8 substitutions: Y27X₆; T28X₂; F29X₁; T30X₄; R67X₄; S98X₂; V68X₁; and R72X₁; optionally with up to four additional framework substitutions, wherein each X₁ residue is independently selected from the group consisting of I, A, L, M, and V; each X₂ residue is independently selected from the group consisting of N, C, Q, S, and T; each X₄ residue is independently selected from the group consisting of K, R, and H; and the X₆ residue is selected from the group consisting of F and W; and
b) the light chain variable domain (VK domain) comprises SEQ ID NO:2; optionally with up to four framework substitutions.

2. An antibody according to claim 1 comprising a heavy chain variable domain and a light chain variable domain, wherein
a) the heavy chain variable domain (VH domain) comprises SEQ ID NO:1 having at least 6 of the following 8 substitutions: Y27F; T28N; F29I; T3OK; R67K; S98N; V68A; and R72A ; optionally with up to four additional framework substitutions; and
b) the light chain variable domain (VK domain) comprises SEQ ID NO:2; optionally with up to four framework substitutions.

3. An antibody according to claim 1 which comprises one, two or three additional heavy chain domain framework substitutions selected from S77X₂, K23X₂, and R38X₄, wherein each X₂ residue is independently selected from the group consisting of N, C, Q, S, and T, and the X₄ residue is selected from the group consisting of R, H, and K.

4. An antibody according to claim 3 which comprises one, two or three additional heavy chain domain framework substitutions selected from S77N, K23T, and R38K.

5. An antibody according to claim 1 which comprises the heavy chain domain framework substitution S98N.

6. An antibody according to claim 1 wherein the light chain domain comprises 1, 2 or 3 substitutions selected from the group 12X₁, V3X₁, and Q50X₄, wherein each X₁ residue is independently selected from the group consisting of V, L, A, I, and M; and the X₄ residue is selected from the group consisting of K, R, and H.

7. An antibody according to claim 6 wherein the 1, 2 or 3 substitutions are selected from I2V, V3L, and Q50K.

8. An antibody according to claim 1 wherein the VH domain comprises SEQ ID NO:1 having the substitutions Y27F; T28N; F29I; T3OK; R67K; V68A; R72A and S98N, together with from one, two or three additional framework substitutions selected from S77N, K23T, and R38K.

9. An antibody according to claim 8 wherein the additional framework substitutions are K23T and S77N.

10. An antibody according to claim 1 wherein the VH domain comprises the HM (SEQ ID NO:3), HN (SEQ ID NO:4), or HQ (SEQ ID NO:5) VH domain.

11. An antibody according to claim 1 wherein the VK domain comprises SEQ ID NO:2.

12. An antibody comprising the VH domain of SEQ ID NO:3 and the VK domain of SEQ ID NO:2.

13. A bispecific antibody comprising a first antigen binding site comprising the heavy chain and light chain variable domains of claim 1 and a second antigen binding site that can bind to another infectious agent, wherein said other infectious agent is optionally the Coxsackievirus A16 (CA16) virus.

14. A pharmaceutical composition comprising an antibody according to claim 1 with a pharmaceutically acceptable carrier.

15. The pharmaceutical composition according to claim 14 further comprising a second antibody that binds another infectious agent, wherein said second antibody is optionally an antibody that can bind to the Coxsackievirus A16 (CA16) virus.

16. A nucleic acid encoding an antibody of claim 1.

17. A vector comprising the nucleic acid of claim 16 operably linked to a promoter.

18. A host cell comprising the nucleic acid of claim 16.

19. A method for making an antibody according to claim 1 the method comprising expressing, in a host cell culture, a vector comprising a nucleic acid encoding an antibody of claim 1 operably linked to a promoter to produce said antibody; and recovering the antibody from the cell culture.

20. A method of treatment or prophylaxis of HFMD by administering, to an individual in need of treatment, an effective amount of an antibody according to claim 1.

21. An antibody according to claim 1 for use in a method of treatment of the human or animal body.

22. An antibody for use according to claim 21 wherein the treatment is therapeutic treatment or prophylactic treatment of HFMD in an individual.

23. A diagnostic kit for detecting the presence of EV71 antigen or the SP70 peptide epitope of the EV71 antigen in a sample of body fluid, wherein the kit comprises an antibody according to claim 1.
